(19) United States
(12) Patent Application Publication
(10) Pub. No.: US 2018/0303935 A1
(43) Pub. Date: Oct. 25, 2018

(54) A NANOMATERIAL COMPLEX COMPRISING GRAPHENE OXIDE ASSOCIATED WITH A THERAPEUTIC AGENT AND METHODS OF USE

(71) Applicant: UNIVERSITY OF UTAH RESEARCH FOUNDATION, Salt Lake City, UT (US)

(72) Inventors: Xinjian Chen, Holiday, UT (US); Chengke Luo, Changsha (CN); Peter Jensen, Salt Lake City, UT (US); Li Lan, Changsha (CN); Alana Welm, Salt Lake City, UT (US)

(21) Appl. No.: 15/768,700

(22) PCT Filed: Oct. 14, 2016
(86) PCT No.: PCT/US16/57060

§ 371 Date: Apr. 16, 2018

Related U.S. Application Data
(60) Provisional application No. 62/315,422, filed on Mar. 30, 2016.
(60) Provisional application No. 62/242,564, filed on Oct. 16, 2015.

(51) Int. Cl. A61K 39/395; A61K 47/02; A61K 47/10; C07K 16/28; C07K 16/32; A61P 35/00

(52) U.S. Cl. CPC A61K 39/39558; A61K 39/3955; A61K 47/02; A61K 2300/00; C07K 16/2887; C07K 16/32; A61P 35/00; A61K 47/10

(57) ABSTRACT

Disclosed herein, are compositions comprising one or more therapeutic agents non-covalently conjugated to a nanomaterial (e.g., graphene oxide). Also, described herein, are methods of preparing stable compositions, the methods comprising a plurality of antibodies non-covalently bound to graphene oxide; physiologically acceptable compositions including them; and methods of administering the compositions to patients for the treatment of a disease such as cancer and autoimmune disorders as well as for the prevention of graft rejection.

### CROSS REFERENCE TO RELATED APPLICATIONS

This application claims the benefit of the filing dates of U.S. Provisional Application Nos. 62/242,564, which was filed on Oct. 16, 2015, and 62/315,422, which was filed on Mar. 30, 2016. The content of these earlier filed applications is hereby incorporated by reference herein in their entirety.

### BACKGROUND

Blood cell cancers include leukemia and lymphomas. Two major types of lymphomas are Hodgkin's lymphoma and non-Hodgkin's lymphoma (NHL). NHL is one of the most common hematologic malignancies among adults, with most types of NHL affecting B cells. Current treatments involve combining chemotherapy with anti-CD20 antibodies. Rituximab, for example, is an anti-CD20 monoclonal antibody considered a first-line therapy for all common B cell malignancies. Rituximab and other monoclonal antibodies are not cytotoxic, but are rather cytostatic, relying on host immune system mechanisms to attack target cells, thus, limiting their therapeutic efficacy when the host effector functions are unavailable or compromised. A significant proportion of patients fail to respond to treatments containing rituximab or relapse after receiving the treatment.

Osteosarcoma is the most common primary malignant tumor of the bone, affecting predominantly children and adolescents. The tumor has a propensity for early invasion and systemic metastases. Among the new patients diagnosed with osteosarcoma in North America, 20% will have clinical evidence of metastatic disease. Radical surgical resection was previously the mainstay therapy but was associated with a high frequency of recurrence and poor prognosis because of early metastasis. Although the use of intensive chemotherapy given before and after surgery has improved the survival of the patients with localized tumor, patients with metastatic disease continue to do very poorly. For patients with non-resettable metastases, the 2-year event-free survival rate is only 15% to 20%.

Pancreatic cancer is the fourth leading cause of cancer death in the US and western countries, and one of the most lethal cancers [15]. Chemotherapy provides minimal survival benefit [16] and combining epidermal growth factor receptor-(EGFR) targeted therapy with chemotherapy does not further improve the outcome of chemotherapy [17]. Taken together, currently, no effective therapies for treatment of pancreatic cancer are available after surgery.

An alternative approach is needed for improving the efficacy of existing therapeutic agents, including antibodies, for the treatment of a variety of diseases and/or conditions including but not limited to blood cell cancers, osteosarcoma, pancreatic cancer, lung cancer, colon cancer, autoimmune disorders and the prevention of graft rejection.

### SUMMARY

Disclosed herein, are compositions comprising one or more therapeutic agents non-covalently conjugated to graphene oxide.

Disclosed herein, are compositions comprising one or more antibodies non-covalently conjugated to graphene oxide.

Disclosed herein, are compositions for delivery of one or more antibodies to a cell, comprising: (a) graphene oxide; and (b) one or more antibodies non-covalently bound to the graphene oxide, wherein the non-covalent binding induces a conformational change in graphene oxide.

Disclosed herein, are kits comprising a composition comprising graphene oxide and one or more therapeutic agents, wherein the graphene oxide is non-covalently bound to the one or more therapeutic agents; and instructions for using the composition.

Disclosed herein, are kits comprising a composition comprising graphene oxide and one or more antibodies, wherein the graphene oxide is non-covalently bound to the one or more antibodies; and instructions for using the composition.

Disclosed herein, are methods of preparing a nanomaterial complex for delivery of a plurality of antibodies to a cell, the method comprising: (a) preparing the nanomaterial through a functionalization process, wherein the nanomaterial is a graphene oxide sheet; (b) attaching a plurality of antibody monomers to the graphene oxide sheet, wherein the loading is through non-covalent binding; (c) incubating the plurality of antibody monomers with the graphene oxide sheet in a low salt solution; and (d) forming a stable aqueous dispersion of the nanomaterial complex.

Disclosed herein, are methods of preparing a nanomaterial complex for delivery of a multivalence of antibodies to a cell, the method comprising: (a) preparing the nanomaterial through a functionalization process, wherein the nanomaterial is a graphene oxide sheet; (b) forming a stable aqueous dispersion of the nanomaterial complex; (c) attaching antibody monomers to the graphene oxide sheet for formation of multivalent antibodies, wherein the loading is through non-covalent binding; (d) incubating antibody monomers with the graphene oxide sheet in a low salt solution.

Disclosed herein, are methods of preparing a nanomaterial complex for delivery of a plurality of antibodies to a cell, the method comprising: (a) preparing the nanomaterial through a functionalization process, wherein the nanomaterial is a graphene oxide sheet; (b) forming a stable aqueous dispersion of the nanomaterial complex; (c) attaching a plurality of antibody monomers to the graphene oxide sheet, wherein the loading is through non-covalent binding; (d) incubating the plurality of antibody monomers with the graphene oxide sheet in a low salt solution; and (d) forming a stable aqueous dispersion of the nanomaterial complex, thereby preparing the nanomaterial complex for delivery of the plurality of antibodies to the cell.

Other features and advantages of the present compositions and methods are illustrated in the description below, the drawings, and the claims.

### BRIEF DESCRIPTION OF THE DRAWINGS

FIGS. 1A-ID show the results of an analysis of the association between rituximab (RTX) and graphene oxide (GO) by their UV-Vis spectrum (A) and SDS Page (B-D). FIG. 1A shows the UV-Vis absorbance curves of free RTX (1000 μg/ml), GO (20 μg/ml), and RTX non-covalently conjugated to GO (designated RTX/GO; 1000/200 μg/ml) are overlaid. FIG. 1B is an SDS PAGE of RTX/GO. RTX of indicated concentration ranging from 200 to 25 μg/ml was loaded in lanes 1-4. RTX/GO thoroughly washed with water, 10%, 50% and normal PBS at room temperature was loaded in lanes 5, 6, 7 and 8. FIG. 1C shows RTX/GO washed with normal PBS at 37° C. for 30 min or overnight (ON), loaded in lanes 4 and 5. FIG. 1D shows RTX and GO incubated at 37° C. overnight with water, 10% and normal PBS; RTX non-covalently conjugated to GO was isolated from each incubation, and loaded in lanes 4, 5 and 6, respectively. HC and LC indicate the heavy and light chain of RTX. Each experiment was repeated at least three times.

FIGS. 2A-2C illustrate the staining of Raji cells using flow cytometry and immunofluorescence microscopy. FIG. 2A shows Raji cells stained with free FITC-RTX (10 μg or 100 μg) and FITC-RTX/GO ((5 μg/ml)/(1 μg/ml)) mixture. FIG. 2B shows CD20-negative Ewin's sarcoma (SKES1) cells stained with FITC-RTX or FITC-RTX/GO. The cells were analyzed by flow cytometry. The experiments were repeated at least three times. FIG. 2C shows Raji cells stained with FITC-RTX and FITC-RTX/GO mixture for 2 hours (upper panel) or 8 hours (lower panel) and examined by immunofluorescence microscopy.

FIGS. 3A-3D demonstrate cytotoxicity of RTX/GO. FIG. 3A shows Raji cells cultured for two days with (from the left to right) PBS control, free RTX at 5 μg/ml, RTX/GO (5 μg/1 μg/ml) or free RTX at 50 μg/ml. FIG. 3B shows the results of flow cytometry analysis of Raji cell cultures: Raji cells were cultured with (from the left to right) PBS control, GO, free RTX or RTX/GO. The upper panel displays FSC by SSC showing that most cells cultured with PBS, GO and RTX are viable, appearing in the live cell gates while the culture with RTX/GO consists mostly of dead cell debris with only few viable cells left in the live cell gate. The histograms in the lower panel show Annexin V levels on the cultured cells. FIG. 3C shows Raji cells cultured overnight with decreasing concentrations of RTX/GO or RTX plus GO (designated RTX+GO) added separately at initiation of the culture, starting from 12.5 μg/ml (RTX) and 2.5 μg/ml (GO). FIG. 3D shows the number of viable Raji cells in culture with RTX/GO followed over time. The results are representative of multiple experiments.

FIGS. 4A-4E illustrate the impact of free RTX and RTX/GO on diffuse large B cell lymphoma cell lines SUDHL-4 (A) and SUDHL-9 (B), Burkitt lymphoma cell line Daudi (C), normal B lymphocytes (D), and peripheral blood mononuclear cells (PBMCs) from a patient with chronic lymphocytic leukemia/lymphoma (CLL) (E). The viable cell numbers plotted are the average of culture triplicates.

FIGS. 5A-5B depict pathological examination of xenograft metastatic Burkitts lymphoma in the livers of pGO (pegylated GO), free RTX and RTX/pGO treated NOD-rag^{-/-}/γ^{-/-} (NRG) mice. FIG. 5A are gross images of livers from pGO (left), RTX (middle), and RTX/pGO (right) treated mice. FIG. 5B are microscopic images of the liver sections from the above mice stained with H&E or CD20 as indicated. Experiments were repeated twice, with three to four mice in each group.

FIGS. 6A-6E illustrate the therapeutic capacity of RTX/GO in vivo. FIG. 6A is an image taken 3 weeks after mixture setup. FIG. 6B, using fluorescence microscopy, shows location of FITC-RTX/GO in the lung and liver. FIG. 6C shows the results of flow cytometry analysis of the bone marrow from xenograft Burkitt lymphoma-bearing mice before or after receiving the indicated treatments. FIG. 6D is a bar graph illustrating Raji cell number per unit volume of bone marrow of indicated treatment groups. FIG. 6E are H&E stained microscopic images (top panel, 4× magnification) showing lymphoma (blue) infiltrate identified in the livers of PBS, GO, and RTX-treated mice but not of RTX/GO treated mice. The lack of lymphoma in RTX/GO-treated mouse liver was confirmed by staining for CD20 (bottom panel).

FIG. 7 shows gel electrophoresis of DNA isolated from Raji cells cultured with PBS, GO, RTX and RTX/GO for 2 days.

FIG. 8 shows the percentage of live Raji cells after overnight culture with RTX/GO, RTX/GO plus Z-VAD-FMK or latrunculin B (LATB).

FIG. 9 is a graph depicting the percentage of live Raji cells in culture with RTX/GO at different RTX:GO ratios.

FIG. 10 is a graph showing the percentage of live Raji cells cultured with 10% PBS, GO, RTX and RTX/GO in the presence of heat-activated fetal bovine serum (FBS), or with RTX/GO in the absence of FBS.

FIGS. 11A-C compares the in vitro potency of various covalent and non-covalent mixtures. FIG. 11A from Sun et al., (2008) Nano Res. 1(3): 203-212) compares the in vitro potency of free doxorubicin (DOX); DOX non-covalently loaded onto a covalent complex of Nano-GO and polyethylene glycol (NGO-PEG/DOX); a mixture of DOX, the antibody Rituximab (Ab), and NGO covalently bound to PEG (NGO-PEG with free DOX and free Ab); and DOX non-covalently loaded onto a covalent complex of NGO, PEG and Ab (NGO-PEG-Ab/DOX). FIG. 11B shows similar in vitro studies after culture with PBS, GO, RTX, and RTX/GO. FIG. 11C contrasts the potency of the non-covalent complex of RTX/GO with free RTX and free GO added together.

FIG. 12 is a graph showing that RTX/GO treatment preserves normal lymphocytes compared to RTX plus the chemotherapeutic drugs, gemcitabine and oxaliplatin.

FIGS. 13A-B illustrate the impact of RTX/GO treatment on survival of mice with Raji cell tumors. FIG. 13A shows one group of mice that received iv RTX/GO twice a week for three and half weeks, or once a week for 10 weeks. FIG. 13B shows, in a separate experiment, Raji cell-grafted mice with transfused human lymphocytes on day 8 and day 10.

FIG. 14 shows GO dispersion in human serum.

FIG. 15 shows the results of high performance liquid chromatography (HPLC) of sonicated/filtered GO (sometimes also referred to as sGO).

FIG. 16 shows the UV-Vis spectrum absorbance of free TRA, GO, TRA non-covalently conjugated to GO (designated TRA/GO) and TRA mixed with GO (designated TRA+GO).

FIG. 17 is an SDS PAGE quantitation of TRA/GO.

FIG. 18 shows MG63 cells, analyzed by flow cytometry, stained with FITC-TRA at 50 μg/ml, or FITC-TRA/GO (50 μg/10 μg/ml) for 2 hours.

FIGS. 19A-B show that TRA/GO is cytotoxic. FIG. 19A shows that TRA/GO is cytotoxic to MG63 cells. FIG. 19B shows that TRA/GO is cytotoxic to HOS cells. * indicates p<0.05 in comparison to PBS. GO and TRA.

FIG. 20 shows that TRA/GO causes non-apoptotic cell death of MG63 cells.

FIG. 21 shows absorbance results (using CCK8) of MG63 cells after overnight culture with TRA/GO, TRA/GO plus Z-VAD-FMK (160 mM) in a bar graph. All the results are representative of multiple experiments. *p<0.05 PBS overnight vs TRA/GO overnight; #p<0.05 TRA/GO or OXA against PBS (3 day); ̂p<0.05 TRA/GO+ZVAD against PBS (3 day); & p<0.05 OXA+ZVAD against PBS (3 day).

FIG. 22 shows that PER/GO is cytotoxic to MG63 cells. * indicates p<0.05 in comparison to PBS, GO and PER.

FIG. 23 shows the lungs of osteosarcoma-bearing mice before and after the treatment with TRA or TRA/GO.

FIG. 24 shows microscopic images of subcutaneous tumors of TRA and TRA/GO treated mice.

FIG. 25 shows the microscopic images of the lungs of TRA and TRA/GO treated mice.

FIG. 26 is a Kaplan-Mere survival curve of the osteosarcoma-bearing mice receiving the indicated treatments.

FIG. 27 shows flow cytometry of BxPC3 cells.

FIG. 28 shows that TRA/GO is cytotoxic to BxPC3 cells, a pancreatic ductal carcinoma cell line. Flow cytometry of BxPC3 cells after four hours of culture demonstrates loss of live cells identified by the LIVE/DEAD cell dyes (top row). The bar graphs show absolute numbers of live cells per LIVE/DEAD cell stain (lower left) and percentage of live cells relative to 10% PBS culture per the CCK-8 Kit assay.

FIG. 29 shows that CTX/GO (CTX non-covalently conjugated to GO) is cytotoxic to colorectal carcinoma cell lines RKO and DLD1. * indicates p<0.05 in comparison to PBS, GO and CTX.

FIG. 30 shows the flow cytometry results of lung carcinoma cell lines, H1944 and H1650, cultured overnight with PBS. GO, free PNT or PNT/GO.

FIG. 31 compares relative tumor volume in immunodeficient NSG mice bearing subcutaneous osteosarcoma treated intravenously with PBS. TRA/GO or Kadcyla®.

FIG. 32 shows lymphocyte counts remain unchanged after overnight culture of human peripheral blood mononuclear cells (PBMC) with PBS or TRA/GO.

FIG. 33 shows that mice treated with a chemo drug plus TRA lost weight while treatment with TRA/GO showed no change in body weight.

FIG. 34 shows Western blot lysates of MG63 cells treated with 10% PBS, TRA, GO, TRA/GO, GO (50 μg) or GO+TRA using phosphor-tyrosine mouse monoclonal antibody (pTyr-100) and glyceraldehyde 3-phosphate dehydrogenase antibody (GAPDH).

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step. The terms “subject,” “individual,” or “patient” are used herein interchangeably.

As used herein, the term “nanomaterial” refers to a carrier structure that is biocompatible with and sufficiently resistant to chemical and/or physical destruction by the environment of its use (e.g., human body, circulatory system) such that a sufficient amount of the nanomaterial remains substantially intact after delivery and reaches its target (e.g., the nucleus of a cell or other cellular structure). Drugs, active agents, therapeutic agents and the like can be incubated or mixed with nanomaterials and then subsequently adsorbed or attached or bonded to the nanomaterial.

As used herein, the term “B cell” refers to a type of immune system cell that comprises a lymphocyte subset that is well-known to be important in the humoral immune response.

As used herein, the term “comprising” can include the aspects “consisting of” and “consisting essentially of.”

As used herein, the phrase “supramolecular bonding” refers to the chemical system involved in the interactions and the assembly of the components that come together in a particular chemical system. The force involved in the spatial organization of a chemical system ranges from weak (e.g., intermolecular forces, electrostatic, and hydrogen bonding) to strong (e.g., covalent bonding). Supramolecular bonding includes non-covalent interactions between molecules involving a wide variety of forces including hydrogen bonding, metal coordination, hydrophobic forces, van der Waals forces, pi-pi interactions and electrostatic effects.

As used herein, the term “crosslinking” means the process of joining two or more molecules together chemically through a covalent bond or other means of attachment.

### Introduction

CD20.

Rituximab (RTX) is a chimeric IgG1 monoclonal antibody (mAb) specific for the B cell associated antigen CD20 (Weiner et al. Seminars in hematology, 2010, 47(2): p. 115-23). As a first-line therapy, RTX is used for treatment of nearly all types of B-cell non-Hodgkin's lymphomas (NHL), including follicular lymphoma, mantle cell lymphoma, diffuse large-B-cell lymphoma (DLBL) and B-cell chronic lymphocytic leukemia (CLL). Despite the success with using RTX as a treatment for NHL, major challenges remain with RTX-based therapy. Only a fraction of patients with low-grade lymphomas respond to RTX monotherapy (Cheson and Leonard, N Engl J Med, 2008, 359(6):613-26). As a result, RTX has been used in combination with chemotherapy. Although combining RTX with chemotherapy improves the therapeutic outcome, a substantial proportion of patients still fail to achieve a complete remission and many relapse (Coiffier, B., et al. Blood, 2010, 116(12):2040-5; and Foa, R., et al., Am J Hematol, 2014, 89(5):480-6).

RTX by itself is not cytotoxic; it is incapable of directly killing the target cells, and only mildly inhibitory to lymphoma cell proliferation in vitro. In the absence of chemotherapy, RTX depends primarily on the indirect, host immune effector functions to kill target cells, likely including antibody-dependent cellular cytotoxicity (ADCC), phagocytosis, and complement-dependent cytotoxicity. Chemotherapy is often associated with serious side effects, including hematologic toxicities resulting in cytopenia and chronic depletion of immune effector cells (Langerbeins, P., et al., Am J Hematol, 2014, 89(12):E239-43), abrogating the mechanism of action of RTX. These therapeutic barriers can be circumvented if RTX and other non-cytotoxic antibodies or therapeutic agents can become cytotoxic with the capacity to kill target cells independent of host effector mechanisms or chemotherapy.

HER2.

Approximately 40-60% of osteosarcomas overexpress HER2, with or without HER2 gene amplification [1-5]. Some studies report a correlation between HER2 expression and poor prognosis for osteosarcoma patients [1, 2, 6] while others did not identify such correlation [3-5]. Despite the controversies, a phase II clinical trial has recently been conducted using an anti-HER2 antibody, trastuzumab (TRA) in combination with cytotoxic chemotherapy for treatment of metastatic HER2-positive osteosarcoma [1]. The result of this trial showed that TRA plus intensive chemotherapy added no appreciable therapeutic benefit as compared to chemotherapy alone, and the outcome for all patients was poor, with no significant difference between the HER2-positive and HER2-negative groups. Therefore, different from breast carcinoma, the anti-HER2 therapy offered no therapeutic benefit to patients with osteosarcoma [7].

HER2 is a transmembrane receptor of the epidermal growth factor receptor (EGFR) superfamily, a receptor tyrosine-protein kinase known to play an important role in promoting growth and metastasis of HER2-positive tumors. HER2 overexpression has been identified in a large variety of human cancers, including, for example, breast carcinoma, osteosarcoma, synovial sarcoma, glioblastoma multiforme and carcinoma of the head and neck, stomach, esophagus, colon, pancreas, lung, endometrium, uterine cervix, ovary, urinary bladder, and retinoblastoma. The anti-HER2 antibodies, trastuzumab (TRA) and pertuzumab (PER), are monoclonal antibodies against HER2 and are FDA approved for the treatment of HER2+ metastatic breast cancer, either in combination with cytotoxic chemotherapy or as monotherapy following prior chemotherapy. While TRA or PER treatment has improved the treatment outcome of HER2+ breast carcinoma, their impact on patient survival remains rather limited. In recent clinical trials, TRA treatment has also been tested for treatment of other HER2+ cancers than of the breast, including osteosarcoma and pancreatic carcinoma. No therapeutic benefit, however, has been obtained. In an attempt to improve therapeutic capacity of anti-HER2 antibodies, the anti-Her2 antibodies as described herein can be conjugated with graphene oxide (GO). The present disclosure reports that the non-covalent conjugation of anti-HER2 antibodies, TRA or PER, to GO confer direct cytotoxicity to HER2+ malignancies, thereby enabling these antibodies to eradicate metastatic tumor in animal models. Similar results have been obtained with pancreatic carcinoma cells.

Pancreatic adenocarcinomas (17%/o to 33%) have also been reported to overexpress HER2 [18-22]. Recent clinical trials, however, have found that TRA treatment was marginally effective against HER2 positive pancreatic adenocarcinoma, even when used in combination with chemotherapy [23, 24]. The result of a recent study using a combination of anti-EGFR and HER2 antibodies suggests a potential therapeutic benefit of such approach for HER2+ pancreatic adenocarcinoma [25].

The majority of pancreatic cancers (85%) are ductal adenocarcinoma, for which the prognosis is extremely poor, making it the most dismal of all the cancers. The American Cancer Society prognosis figures show that the five-year survival rate of pancreatic cancer is about 4% [15]. The extremely grim prognosis is attributed to the tendency of the tumor to undergo early local invasion and distal metastasis. Only about 15% of patients with pancreatic cancer are found to be eligible for surgery. Even with patients who receive radical surgical resection by highly experienced surgeons, only about 20% would survive 5 years, with a median survival of 15.5 months.

EGFR.

Anti-EGFR1 (epithelial growth factor receptor) antibodies cetuximab (CTX) and panitumumab (PNT) have been used in combination with chemotherapy as a standard therapy for treatment of metastatic colorectal carcinoma harboring wild-type KRAS genes [27, 28]. Despite the improvement in patient response, disease control and survival, the therapeutic benefit with the adjuvant anti-EGFR1 antibody treatment is rather limited: adding the antibody to the chemo regimen only prolongs the survival for a few months. In addition, the antibody therapy provides no benefit to patients whose tumors have mutated KRAS genes.

The present disclosure features compositions comprising one or more therapeutic agents, such as RTX. CTX, PNT. TRA, or PER, non-covalently conjugated to a nanomaterial (e.g., graphene oxide). As used herein, the term “RTX/GO” refers to RTX non-covalently conjugated to grapheme oxide (GO). Similarly, “CTX/GO,” “PNT/GO,” “TRA/GO,” and “PER/GO” refer to CTX, PNT, TRA, or PER non-covalently conjugated to GO, respectively. For example, disclosed herein are compositions and methods of generating multivalent RTX with the capacity to crosslink CD20 using a recently characterized nanomaterial, graphene oxide. Also, disclosed herein are compositions and methods of generating multivalent CTX, PNT, TRA and PER. The present disclosure, also includes therapeutic agents including antibodies such as, for example, RTX, CTX. PNT, TRA, PER, that can be stably and non-covalently associated (e.g., bound or conjugated) with GO that are, or can subsequently be converted to, cytotoxic therapeutic agents capable of killing the targeted cells, for example, malignant B cells. As described herein, when therapeutic agents (e.g., antibodies) are non-covalently associated with GO, they can be cytotoxic therapeutic agents capable of killing targeted cells, for example, malignant B cells. In some aspects, the cytotoxicity of the therapeutic agents can be attributed to GO, the therapeutic agent or both.

Compositions

Therapeutic Agents.

As used herein, the term “therapeutic agent” refers to any agent having a therapeutic effect, including toxic agents, such as, for example, chemotherapeutics, and non-toxic material (e.g., nanomaterial, such as graphene oxide). A therapeutic agent can be a peptide, protein, an antibody, an antibody fragment or a commercially known drug. A variety of therapeutic agents including cytotoxic and cytostatic agents can be incorporated into the compositions described herein.

In an aspect, the therapeutic agent is a cytostatic agent or non-toxic agent that is subsequently converted to a cytotoxic agent using the methods described below. In an aspect, the composition described herein can comprise one or more therapeutic agents non-covalently conjugated to graphene oxide, wherein one or more of the therapeutic agents is cytostatic prior to its association with graphene oxide.

In some aspects, the compositions comprise one or more therapeutic agents (e.g., rituximab, obinutuzumab, blinatumomab, trastuzumab, pertuzumab, cetuximab or panitumumab), including but not limited to antibodies or biologically active variants thereof. For example, if the therapeutic agent is an antibody, the antibody can be a single chain antibody (scFv) or a Fab fragment; a human, chimeric or humanized antibody or a biologically active variant thereof; and/or can be (or can be derived from) a monoclonal or polyclonal antibody. The antibody can be a naturally expressed antibody (e.g., a tetrameric antibody) or a biological variant thereof.

In some aspects, the therapeutic agent can be a non-naturally occurring antibody (e.g., a single chain antibody or diabody) or a biologically active variant thereof. As noted above, the variants include, without limitation, a fragment of a naturally occurring antibody (e.g., a Fab fragment), a fragment of a scFv or diabody, or a variant of a tetrameric antibody, an scFv, a diabody, or fragments thereof that differ by an addition and/or substitution of one or more amino acid residues. The antibody can also be further engineered.

In some aspects, the compositions, described herein, comprise one or more therapeutic agents non-covalently bound to a nanomaterial (e.g. GO), wherein the therapeutic agent is an antibody that binds B cells or a B lymphocyte antigen (e.g., CD20. CD19, CD3, CD22 or CD38). CD20 is expressed on the surface of all B cells during most phases of B cell development. CD20 is not expressed on early pro-B cells or plasma blasts and plasma cells, while CD19 is expressed on early pro-B cells and early plasma cells. CD20 is a glycosylated phosphoprotein, the expression of which is activated during pro-B phase with the level of expression increasing during B cell maturation. Flow cytometry as well as immunocytochemistry techniques can be carried out to detect its presence on tumors. The presence of CD20 on B cells can indicate or assist in diagnosing a patient with a disorder or disease associated with the expression of CD20. Studies have shown that CD20 is expressed on B cell lymphomas, hairy cell leukemia, B cell chronic lymphocytic leukemia, melanoma cancer stem cells, Hodgkins lymphoma, myeloma, pancreatic carcinoma and thymoma. Accordingly, in some embodiments, the therapeutic agent binds CD20. In other aspects, the therapeutic agent is capable of crosslinking to a target, such as a CD20 antigen. CD19 is a transmembrane glycoprotein and a member of the immunoglobulin superfamily that is expressed on the surface of leukemia cells, and more specifically, acute lymphoblastic leukemia. Studies have shown that CD19 is also expressed on tumor cells with both B-cell non-Hodgkin's lymphoma and chronic lymphocytic leukemia. The presence of CD19 on B cells can indicate or diagnose a patient with a disorder or disease associated with the expression of CD19. For example, studies have shown that CD19 expression is associated with B cell lymphomas. CD22 is a transmembrane protein expressed on acute lymphoblastic leukemia cells. CD38 is a surface protein that is expressed by most, if not all, multiple myeloma cells.

In some aspects, the compositions, described herein, comprise one or more therapeutic agents non-covalently bound to a nanomaterial (e.g., GO), wherein the therapeutic agent is an antibody that binds to one or more of ErbB family of receptors (also referred to as HER family of protein-tryosine kinases). The ErbB lineage is structurally similar to the epidermal growth factor receptor (EGFR), and includes HER1 (EGFR EGFR1, ErbB1), HER2 (Neu, ErbB2), HER (ErbB3), and HER4 (ErbB4). HER1 and HER2 can be overexpressed in cancer, and is associated with a negative prognosis. For example, studies have shown that HER1 is overexpressed in colorectal and lung cancers, while HER2 overexpression is associated with osteosarcoma, pancreatic, breast, testicular, gastric and esophageal cancers. Accordingly, in some embodiments, the therapeutic agent binds HER1. In other aspects, the therapeutic agent binds HER2.

In some aspects, the therapeutic agent can be a monoclonal antibody. In an aspect, the monoclonal antibody targets CD20 or CD19 (i.e., the therapeutic agent is an anti-CD20 or anti-CD19 monoclonal antibody). Examples of anti-CD20 monoclonal antibodies include but are not limited to rituximab (also referred to as Rituxan, MabThera and Zytux), obinutuzumab (also referred to as Gazyva® and Gazyvaro), ibritumomab tiuxetan (also referred to as Zevalin®), tositumomab (also referred to as Bexxar®), ofatumumab (also referred to as Genmab® and HuMax-CD20), AME-133v, and IMMU-106 or a biologically active variant thereof. Examples of anti-CD19 monoclonal antibodies include blinatumomab (also referred to as Blincyto®, and AMG103). GBR 401, coltuximab ravtansine (also known as SAR3419), denintuzumab mafodotin (also known as SGN-CD19A and SGN-19A) and taplitu-momabpaptox or a biologically active variant thereof. In an aspect, the monoclonal antibody targets an ErbB family of receptors (e.g., HER1, HER2). Examples of anti-HER1 monoclonal antibodies include but are not limited to cetuximab (also referred to as Erbitux®), and panitumumab (also referred to as ABX-EGF, Vectibix®) or a biologically active variant thereof. Examples of anti-HER2 monoclonal antibodies include but are not limited to trastuzumab (also referred to as Herclon® and Herceptin®) and pertuzumab (also referred to as 2C4 and Perjeta®) or a biologically active variant thereof. In an aspect, the monoclonal antibody targets CD22 or CD38 (i.e., the therapeutic agent can be an anti-CD22 or anti-CD38 monoclonal antibody). An example of anti-CD22 monoclonal antibodies includes, but is not limited to inotuzumab ozogamicin (also referred to as CMC-544). An example of anti-CD38 monoclonal antibodies includes, but is not limited to daratumumab (also referred to as Darzalex®).

In an aspect, the compositions, disclosed herein, comprise one or more therapeutic agents non-covalently bound to a nanomaterial (e.g., GO), wherein the therapeutic agent is an antibody that binds CD3. Examples of anti-CD3 monoclonal antibodies include but are not limited to muromonab-CD3 (also referred to as Orthoclone OKT3®), otelixizumab (also referred to as TRX4), teplizumab (also referred to as MGA031 and hOKT3γ1), and visilzumab (also referred to as nuvion) or a biologically active variant thereof.

Examples of antibodies that can be used in the compositions and methods disclosed herein, include, but are not limited to rituximab, obinutuzumab, ibritumomab tiuxetan, ofatumumab, AME-133v, IMMU-106, blinatumomab, GBR 401, coltuximab ravtansine, denintuzumab mafodotin, taplitu-momabpaptox, cetuximab, panitumumab, trastuzumab, pertuzumab, inotuzumab ozogamicin, daratumumab, muromonab-CD3, otelixizumab, teplizumab, and visilzumab or a biologically active variant thereof.

In some aspects, the one or more therapeutic agents (e.g., one or more antibodies or one or more monomers) can be joined together to form a larger molecule or a polymer of therapeutic agents (e.g., a plurality of one or more therapeutic agents, such as one or more antibody molecules linked or bound together). In some aspects, the one or more therapeutic agents in the form of a polymer can be further non-covalently bound to graphene oxide to form multivalent large molecules (e.g., multivalent antibodies). In an aspect, multiple antibodies in the form of a multimer can be non-covalently bound to graphene oxide to form multivalent large molecules (e.g., multivalent antibodies).

In an aspect, the one or more therapeutic agents, as described herein, comprises one or more monomers. For example, the monomer can be an antibody. In an aspect, the one or more monomers are non-covalently bound to the graphene oxide thereby forming a polymer of therapeutic agents.

The therapeutic agent can be an anti-cancer agent or anti-tumor agent. The anti-cancer agent can be a therapeutic, agent or drug that has anti-cancer properties. Examples of anti-cancer drugs include but are not limited antiproliferative agents, cytotoxic agents, immunosuppressive agents, anti-tumor antibodies, or any anti-cancer agent or any derivatives and/or analogues thereof providing an additional therapeutic benefit. Examples of anti-cancer antibodies include but are not limited to blinatumomab (Blincyto®), brentuximab (Adcetris®), trastuzumab (Herceptin®), pertuzumab (Perjeta®), cetuximab (Erbitux®), and panitumumab (Vectibix®).

In some aspects, the therapeutic agent can be a peptide, protein, an antibody, an antibody fragment, a commercially known drug or biological variant thereof that can be used to treat autoimmune disorders such as multiple sclerosis, rheumatoid arthritis and systemic lupus erythematosis, as well as to prevent or minimize an immune system response due to transplantation of an organ, tissue or cell.

Also disclosed are cells comprising one or more therapeutic agents (e.g., an antibody) non-covalently bound or conjugated to graphene oxide.

Immunomodulators.

In some aspects, the compositions disclosed herein can further comprise one or more immunomodulators. As used herein, the terms “immunomodulator” and “immune modulating agents” refer to a component (e.g., a protein, peptide, pharmacological and/or immunological agent) that modifies (e.g., potentiates) the immune system response toward a desired immune system response. An immunomodulator can also be an adjuvant. The immunomodulator can be a therapeutic agent that specifically or nonspecifically augments an immune system response. The immunomodulators described herein can be provided either alone or in combination with the one or more therapeutic agents non-covalently conjugated to a nanomaterial. The immunomodulator can be covalently or non-covalently conjugated to a nanomaterial (e.g., graphene oxide). Examples of immunomodulators or immune modulating agents include but are not limited to cytokines, interleukins, chemokines or any protein, peptide, pharmacological or immunological agent that provides an increase in an immune system response. In an aspect, the immune modulating agent is a type 1 interferon such as interferon alpha or interferon beta. Other immune modulating agents include but are not limited to anti-CD40 ligand antibody, Flt3 ligand, CD200, TGFβ, PDL1, PDL2, soluble CD83, OX40L, anti-IL-17 antibody, IL-2, IL-10, IL-12, IL-19, IL-33, galectin-1, CTLA-4, CD103 and indoleamine 2,3-dioxygenase.

The immunomodulator can be conjugated to the same graphene oxide sheet as the one or more therapeutic agents or to a separate and different graphene oxide sheet.

Labels.

The compositions as described herein can also include a detectable label. For example, disclosed herein are molecular probes, comprising a composition comprising one or more therapeutic agents non-covalently conjugated to graphene oxide. The phrase “detection label” as used herein refers to any molecule that can be associated with the compositions described herein, directly or indirectly, and which results in a measurable, detectable signal, either directly or indirectly. For instance, the label can be attached to one or more of the therapeutic agents. In an aspect, a molecular probe comprising a composition described herein, further comprises a detectable label.

Examples of detectable labels include fluorescent, radioactive isotopes, fluorescent molecules, phosphorescent molecules, enzymes, antibodies, and ligands. Examples of fluorescent labels include, but are not limited to SYBR Green I (Invitrogen), fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF. Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO). Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3. Fluorescamine, Fura-2. Genacryl Brilliant Red B, Genacryl Brilliant Yellow 10GF, Genacryl Pink 3G, Genacryl Yellow 5GF. Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200). Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 311, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123. Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G. Sevron Brilliant Red B. Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN. Thioflavin 5, Thiolyte, Thiozol Orange, Tinopol CBS, True Blue, Ultralite, Uranine B, Uvitex SFC, Xylene Orange, and XRITC. Fluorescent labels can be obtained from a variety of commercial sources, including Invitrogen, Carlsbad, Calif.; Amersham Pharmacia Biotech, Piscataway, N.J.; Molecular Probes, Eugene, Oreg.; and Research Organics, Cleveland, Ohio.

Nanomaterial.

The nanomaterial disclosed herein generally refers to particles with diameters or size ranging usually from 0.1 to 220 nm. In some aspects, the nanomaterial can be a fullerene (e.g., graphene sheets, quantum dots, nanowires and nanorods). In some aspects, the nanomaterial is graphene. Graphene is comprised of a single layer of carbon. Graphene can be naturally occurring or synthetic. In some aspects, graphene sheets can be used to prepare graphene oxide. Accordingly, in some aspects, the nanomaterial is graphene oxide.

As disclosed herein, the nanomaterial (e.g., graphene oxide) can be functionalized. For example, the graphene oxide can be processed (e.g., sonication and filtration) in such a way that the graphene oxide maintains its inherent properties and reduces precipitation. In some aspects, sonication can be used to break the graphene oxide into small nanosheets that then can be subsequently filtered (e.g., using a 0.22 μm filter). Filtered graphene oxide is considered stable, for example, because it does not precipitate in serum. Other methods of functionalizing nanomaterials can be used and are known to those skilled in the art (for example, see, Yang et al., Nat Protoc., 2013, 8(12):2392-403).

The term “functionalized” can refer to the addition of a solubilizing material, such as, for example, a hydrophilic polymer. For example, functionalized can include the process of pegylation. Therapeutic agents, for example, including those containing aromatic rings can be bonded to the surface of the graphene, grapheme or GO sheets through supramolecular bonding, and pegylation. Pegylation is the process of attaching a polymer (e.g., polyethylene glycol (PEG)-amine) to a molecule (e.g., GO or a therapeutic agent). This process can lead to physical and/or chemical property changes including but not limited to conformation, binding and hydrophobicity. Processes of pegylation are known to one skilled in art (for example, see, Yang et al., Nat Protoc. 2013 December; 8(12):2392-403). Further, the advantages of pegylation are well known and include but are not limited to improved solubility of a therapeutic agent, reducing toxicity, increasing bioavailability, increasing stability, reducing proteolytic degradation, reducing immunogenicity and enhancing clearance from the body of a therapeutic agent.

In some aspects, the graphene oxide is pegylated. For example, graphene sheets (or graphene oxide) can be further linked to, bonded to or associated with, for instance, a hydrophilic polymer such as PEG using known techniques to one skilled in the art (for example, see, Yang et al., Nat Protoc., 2013, 8(12):2392-403). In an aspect, the compositions described herein can further comprise a polymer (e.g., PEG). The polymer can be hydrophilic. In some aspects, graphene oxide can be covalently, or through supramolecular bonding, attached to the hydrophilic polymer (i.e., polymers containing polar or charged function groups rendering them soluble in water). In some aspects, PEG can improve the solubility of GO. In some aspects, PEG can also improve the stability of GO. For example, polyethylene glycol can be coupled to a phospholipid. In such cases, it can be amine-terminated, wherein the solubilizing material can have a hydrophobic portion and a hydrophilic portion. PEGs can have different geometries (e.g., multi-arm, branches, stars or combs). For example, PEG-amine, a two-armed non-branching polymer with a molecular weight of 2,000 can be used. Branched hydrophilic polymers are associated with improving in vivo circulation. Branches can lead to a multivalent complex such that one or more therapeutic agents (e.g., multiple or a plurality of antibodies) can be linked or associated with a nanomaterial (e.g., graphene oxide). For example, PEG or other hydrophilic polymers can be linked at the opposite end from the amine coupling to attach another agent to target a particular cell type. The addition of PEG can increase the solubility and/or stability of GO. In an aspect, multiple therapeutic agents are bonded to a nanomaterial. For example, when multiple therapeutic agents are bonded to a nanomaterial, the therapeutic agents can be the same, different or a combination of the same and different therapeutic agents. In an aspect, the compositions disclosed herein can comprise one or more therapeutic agents non-covalently bound to a nanomaterial (e.g., graphene oxide), and the polymer can be further conjugated to one or more therapeutic agents, such as one or more immunomodulators. For example, interferon alpha can be conjugated to a nanomaterial (e.g., GO) along with a therapeutic agent, such as rituximab.

In some aspects, graphene sheets (or graphene oxide) can be further non-covalently conjugated to one or more therapeutic agents. Accordingly, in some aspects, the compositions as disclosed herein comprise one or more therapeutic agents. In an aspect, the one or more therapeutic agents are non-covalently bound to the graphene oxide forming a nanomaterial complex. The formation of a nanomaterial complex can result from an association of multiple antibody molecules non-covalently bound to individual graphene sheets (or graphene oxide).

In some aspects of the claimed compositions and methods described herein, GO can act as a therapeutic agent, an anti-cancer agent, a target for cancer or tumor cells or can enhance the efficacy of the compositions or other therapeutic agents described herein.

In some aspects of the claimed compositions and methods described herein, GO can act as a therapeutic agent, an anti-cancer agent, or can enhance the efficacy of the compositions or other therapeutic agents described herein.

Conjugation.

As used herein, the terms “associated.” “conjugated,” and “bound” can be used interchangeably unless otherwise explicitly stated. A feature of the present disclosure is the conjugation of compositions comprising one or more therapeutic agents to a nanomaterial (e.g., graphene oxide). A method of conjugating a therapeutic agent to a nanomaterial can rely on or involve supramolecular chemistry. In some aspects, the interaction can be between the therapeutic agent and the nanomaterial rather than within a single component. In an aspect, the therapeutic agent is non-covalently bound (or conjugated) to the nanomaterial. In an aspect, the non-covalent binding is through pi-stacking, a hydrophobic interaction, ionic binding or hydrogen binding. “Pi-stacking” as used herein refers to non-covalent interactions between aromatic rings.

The composition as disclosed herein can also comprise one or more therapeutic agents that are non-covalently conjugated to graphene oxide thereby resulting in a composition that is multivalent. The therapeutic agent:nanomaterial ratio can also be 2:1, 3:1, 4:1, 5:1 or 10:1 or any other combination thereof. In some aspects, the ratio of the one or more therapeutic agents to graphene oxide can range between 1250:1 to 250:1 to 25:1 to 1:1 to 0.2:1. In an aspect, the therapeutic agent and graphene oxide can be present in ratio of 5:1. In some aspects, the mass ratio of the therapeutic agent:nanomaterial is 2:1, 3:1, 4:1, 5:1 or 10:1. In some aspects the molar ratio of therapeutic agent:nanomaterial is 1:1.

The present disclosure also features compositions comprising one or more therapeutic agents such as one or more anti-CD20, anti-CD19, anti-CD3, anti-CD38, anti-CD22, anti-HER1, or anti-HER2 monoclonal antibodies non-covalently conjugated to a nanomaterial (e.g., graphene oxide) wherein the composition has increased affinity for its antigen (e.g., CD20, CD19. CD3, CD38, CD22) or a receptor (e.g., HER1, HER2, T-cell receptor). Such compositions included herein have increased avidity for the antigen (or receptor) for example as much as 10-fold or more. The increased avidity of such compositions, for example, non-covalent conjugation of rituximab, blinatumomab, cetuximab, panitumumab, trastuzumab or pertuzumab to graphene oxide, can be due to the multivalent nature of the composition (e.g., multiple antibody molecules associated with each graphene nanosheet).

As mentioned above, in some aspects, the compositions can comprise one or more therapeutic agents associated with or bound to the nanomaterial (e.g., graphene oxide) through non-covalent interactions including pi-stacking, hydrophobic interactions, ionic binding or hydrogen binding. The compositions described herein can be used to deliver one or more therapeutic agents to a patient for the treatment of cancer. Where two or more different therapeutic agents are non-covalently bound to the nanomaterial, the two or more different therapeutic agents can be referred to as a “first agent,” a “second agent,” a “third agent.” and so on. Useful therapeutic agent combinations can include, for example, antibodies that recognize HER2 through binding to a different epitope or antibodies that bind or target two or more different antigens or receptors. In an aspect, a first agent can be trastuzumab and a second agent can be pertuzumab. In another aspect, a first agent can be trastuzumab and a second agent can be certuximab.

Methods of Delivery of a Therapeutic Agent to a Cell

As used herein, the term “nanomaterial complex” refers to a composition comprising at least one or more therapeutic agents non-covalently conjugated to a nanomaterial (e.g., graphene oxide). “Nanomaterial complex” also refers to the different variations of the compositions described herein. In some aspects, the disclosure features a nanomaterial complex as described above for delivery of one or more therapeutic agents to a cell, comprising a) a nanomaterial having a sp2-hybridized carbon rings with hydroxyl and carboxyl groups; and (b) one or more therapeutic agents attached to the surface of the nanomaterial through supramolecular bonding. In some aspects, the nanomaterial complex is multivalent. In some aspects, the therapeutic agent can be bound to the nanomaterial through pi stacking (i.e., non-covalent interactions between aromatic rings). For example, the therapeutic agent (e.g., antibody) can be non-covalently bound to the nanomaterial, or adsorbed to it by supramolecular chemistry, such as hydrophobic forces or pi-stacking.

Alternatively, the nanomaterial complex as described herein can be formulated for delivery of one or more therapeutic agents to a cell, comprising graphene oxide; and one or more therapeutic agents non-covalently bound to the graphene oxide, wherein the non-covalent binding induces a conformation change in GO. GO is comprised of thin nanomaterial sheets which are comprised of an aromatic monomolecular layer of carbon. In an aspect, the one or more therapeutic agents are a plurality of monomers. As described in the Examples, the non-covalent binding of rituximab, for instance, to GO, can induce the GO sheet to fold up and wrap around the rituximab molecules. This change results in a stable relationship between the antibody (e.g., rituximab, trastuzumab, pertuzumab, cetuximab, panitumumab) non-covalently associated with GO.

The therapeutic agents to be delivered using the materials described herein can comprise a fused aromatic ring structure permitting pi-stacking to the aromatic structure of the nanomaterial.

The nanomaterial complexes as described herein can comprise a hydrophilic polymer attached to the nanomaterial (e.g., graphene oxide), wherein the attachment is through supramolecular bonding of a therapeutic agent to the surface of the nanomaterial thereby forming a stable aqueous dispersion of the complex.

In an aspect, the disclosure features methods for delivering one or more therapeutic agents to a cell (e.g., on a cell), comprising a nanoparticle complex as described herein, the method comprising contacting a cell (or cells) with a nanomaterial complex as described herein for a sufficient time to permit crosslinking of the specific cell surface molecules (e.g. CD20 in the instance of rituximab) on the cell. In an aspect, a hydrophilic polymer is bound to the nanomaterial; and one or more of the therapeutic agents comprising an aromatic molecule non-covalently conjugated to the surface of the nanomaterial (e.g., a graphene sheet or GO). In some aspects, the nanomaterial complex comprises one or more therapeutic agents (e.g., antibodies non-covalently conjugated to the graphene oxide).

In an aspect, the aromatic rings, charged motif and hydrophobic side chains of the one or more therapeutic agents can interact with the nanomaterial (e.g., graphene oxide) through an ionic bond, hydrophobic bond and pi-stacking. Multiple therapeutic agents or a plurality of antibody monomers can be non-covalently bound to the surface of the nanomaterial (e.g., a graphene sheet or GO).

The therapeutic agent can also serve as a targeting agent. In an aspect, the delivery of the nanomaterial complex as described herein can be targeted to certain cell types (e.g., cells expressing CD20 antigens or specific receptors) by attaching (e.g., conjugating) targeting agents (e.g., anti-CD20, anti-CD19, anti-CD3, anti-CD22, anti-CD38, anti-HER1 or anti-HER2 antibodies) associated with the nanomaterial (e.g., graphene oxide) to the cell surface. The cells to be contacted can be tumor cells including malignant cells. Further, the nanomaterial complex can crosslink CD20 on the malignant B cells, thereby leading to cell death.

Described herein are, also cells comprising any one of the compositions described herein.

The method described herein features therapeutic agents such as, for example, antibodies having stable association with the nanomaterial at normal pH (e.g., pH 7.0-7.4). In an aspect, the method further comprises the step of contacting the nanomaterial complex with serum, wherein the nanomaterial complex has a stable association with the nanomaterial in the serum.

The method also features therapeutic agents (e.g., antibodies) having an increased dissociation at low or high pH. In an aspect, the method further comprises the step of contacting the nanomaterial complex with serum, wherein the nanomaterial complex does not dissociate in the serum.

The disclosure also features compositions for delivery of one or more therapeutic agents to a cell, the composition comprising graphene oxide and one or more therapeutic agents non-covalently conjugated to the graphene oxide. The nanomaterial can be graphene oxide or a graphene sheet. In an aspect, the non-covalent interaction induces a conformational change in graphene oxide. Such conformational change can stabilize the non-covalent association of the one or more therapeutic agents to graphene oxide.

Methods of Making Nanomaterial Complexes

Disclosed herein are techniques that can be used to produce the nanomaterial complexes described herein.

For example, the nanomaterial complex described herein can generally be produced by the following steps. First, the nanomaterial (e.g., graphene oxide) can be prepared from graphene using a modified Hummers method. The nanomaterial can then be sonicated to break the graphene oxide into small nanosheets followed by filtration (e.g., filtration can be carried out using a 0.2 μm filter). Alternatively, the nanomaterial complex can be prepared by carrying out surface functionalization (e.g., pegylation) or improved oxidation of graphene (Marcano, et al., (2010), ACS Nano 4:4806-4814) as described therein.

The next step can involve generating cytotoxic therapeutic agents or compositions. For example, static antibodies, such as anti-CD antibodies or other antibodies specific for the determinants overexpressed on tumor cells (e.g., anti-ErbB antibodies), can be converted to antibodies that are cytotoxic. For this, one method that can be used is addition of antibody valences. For example, one or more therapeutic agents (e.g., antibodies) are attached to individual GO nanosheets (GO) to form a polymer of therapeutic agents non-covalently conjugated to GO. More specifically, the polymers of therapeutic agents can be made by loading multiple antibody monomers to graphene oxide nanosheets. The loading can be performed through non-covalent interaction between the antibody molecules and graphene oxide. For instance, the graphene oxide can be incubated with the therapeutic agent (e.g., rituximab, blinatumomab, trastuzumab, pertuzumab, cetuximab, panitumumab) in water or 10% phosphate buffered saline PBS (containing 0.09% NaCl) at 37° C. under constant agitation for a period of time ranging from an hour to overnight. Stable binding between the antibody and the graphene oxide can be achieved by incubating the antibody with the graphene oxide in water or in a low salt solution (10% phosphate buffered saline PBS). Such stable binding between the antibody and GO can prohibit the antibody from being washed away from GO. The binding between the antibody and the graphene oxide that is formed in physiological salt concentration can be unstable and washed from GO. The loading of the antibody to graphene oxide does not impair the antibody (e.g., rituximab, blinatumomab, trastuzumab, pertuzumab, cetuximab, panitumumab) binding capacity to the specific antigen (e.g., CD20, CD19, CD3, CD38, CD22) or receptor (e.g., HER1, HER2, T-cell receptor). In some aspects, the present disclosure features a method of preparing a nanomaterial complex for delivery of a plurality of antibodies to a cell, the method comprising: (a) preparing the nanomaterial through a functionalization process, wherein the nanomaterial is a sonicated, pegylated or super-oxidized graphene oxide sheet; (b) attaching a plurality of antibody monomers to the graphene oxide sheet, wherein the loading is through non-covalent binding; (c) incubating the antibody monomers with the graphene oxide sheet in a low salt solution; and (d) forming a stable aqueous dispersion of the nanomaterial complex. In an aspect, antibody monomers can be mixed with the grapheme oxide sheet in buffered low salt solution. The non-covalent binding can be through pi-stacking or other types of non-covalent interactions as described herein. In an aspect, the antibody monomers can be incubated with the graphene oxide sheet in a low salt solution at 37° C. under constant agitation. In an aspect, the low salt concentration is 10% PBS (containing 0.09% NaCl). In some aspects, the antibody to graphene oxide are present in a mass ratio of 5:1.

The design and delivery of the nanomaterial complexes described herein should take into account the biocompatibility of the nanomaterial. The physical parameters of a nanomaterial can be optimized, with the desired effect governing the choice of size, shape and material. The nanomaterial can also be used as a delivery vehicle and can serve as a scaffold.

The nanomaterial complexes can be prepared in various forms for drug delivery. For instance, the nanomaterial complexes can be prepared as a stable aqueous dispersion, and/or in unit dosage form. In an aspect, methods for preparing a nanomaterial complex for delivery of a small molecule active agent onto a cell, where one first prepares nanomaterial in a suitable form, e.g., in dispersed form suitable for in vivo administration, is disclosed. The materials described herein can exist in dispersed form as opposed to aggregates, which can form in the preparation of hydrophobic nanomaterial.

In some aspects, graphene can be prepared as single atom-thick molecular sheets. The sheets can be controlled in width and length and can be less than 100 nm, 20 nm or less than 10 nm on a side. And, in all cases a size suitable for in vivo administration. Methods of preparing nanomaterials, such as, for example, graphene oxide, are known to one skilled in the art (for example, see, Long Zhang et al., (2009) Carbon 2009, 47:3365-3368).

Methods for stably loading a therapeutic agent (e.g., rituximab, blinatumomab, trastuzumab, pertuzumab, cetuximab, panitumumab) onto graphene oxide are disclosed herein (also, see e.g., Examples 1 and 7). The method can comprise the steps of incubating a therapeutic agent with graphene oxide in a 10% phosphate buffered saline solution for a time period ranging from an hour to overnight.

Antibodies.

As noted above, the nanomaterial complexes as disclosed herein, can include an antibody or a biologically active variant thereof. As is well known in the art, monoclonal antibodies can be made by recombinant DNA. DNA encoding monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

In vitro methods are also suitable for preparing monovalent antibodies. As it is well known in the art, some types of antibody fragments can be produced through enzymatic treatment of a full-length antibody. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking an antigen. Antibodies incorporated into the present composition can be generated by digestion with these enzymes or produced by other methods.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv. Fab, Fab′, or other antigen binding portion of an antibody) which contains a portion of an antigen binding site from a non-human (donor) antibody integrated into the framework of a human (recipient) antibody.

The Fv region is a minimal fragment containing a complete antigen-recognition and binding site consisting of one heavy chain and one light chain variable domain. The three CDRs of each variable domain interact to define an antigen-binding site on the surface of the Vh-Vl dimer. Collectively, the six CDRs confer antigen-binding specificity to the antibody. As well known in the art, a “single-chain” antibody or “scFv” fragment is a single chain Fv variant formed when the Vh and Vl domains of an antibody are included in a single polypeptide chain that recognizes and binds an antigen. Typically, single-chain antibodies include a polypeptide linker between the Vh and Vl domains that enables the scFv to form a desired three-dimensional structure for antigen binding.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies can also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody.

Methods for humanizing non-human antibodies are well known in the art. For example, humanized antibodies can be generated by substituting rodent CDRs or CDR sequences for the corresponding sequences of a human antibody. Methods that can be used to produce humanized antibodies are also well known in the art.

Configurations.

Each part of a given nanomaterial complex described herein, including the therapeutic agent, polymer and nanomaterial, can be selected independently. One of ordinary skill in the art would understand that the component parts need to be associated in a compatible manner. The nanomaterial complexes disclosed herein can be used to deliver therapeutic agents to a patient for the treatment of cancer, an autoimmune disease or disorder or to prevent or reduce symptoms of graft rejection. The therapeutic agent can be comprised of one or more therapeutic agents (e.g., antibodies). For instance, a nanomaterial complex having multiple therapeutic agents and/or a detectable label and/or an immunomodulator and thus, can comprise a “first agent,” and a “second agent” and so on. The compositions described herein can be a combination therapy for a disease (e.g., a cancer). Thus, the nanomaterial complex as disclosed herein can deliver two or more different therapeutic agents (or one type of therapeutic agent combined with a detectable label) or two or more molecules of the same therapeutic agent, that can also include a detectable label and/or an immunomodulator. With the inclusion of a detectable label, the nanomaterial complex as described herein can also be used to map the distribution of targets to which any of the therapeutics can bind. The number of therapeutic molecules per nanomaterial can vary depending on the type of nanomaterial selected.

Accordingly, in some aspects, the therapeutic agent can be two or more. In some embodiments, the therapeutic agent and nanomaterial are present in a ratio (e.g., a mass ratio) of 1:1 (therapeutic agent:nanomaterial). The therapeutic agent:nanomaterial ratio (e.g., a mass ratio) can also be 2:1, 3:1, 4:1 or 5:1 or any other combination thereof. For example, the therapeutic agent:nanomaterial ratio can range between 1250:1 to 250:1 to 25:1 to 1:1 to 0.2:1. The ratio may also vary with therapeutic agents or individual antibodies.

In some aspects, the nanomaterial complex comprises one or more therapeutic agents non-covalently bound to the nanomaterial. The nanomaterial complex can further comprise a polymer such as PEG covalently conjugated to the nanomaterial. In an aspect, the nanomaterial can be functionalized using PEG or super-oxidation.

In addition, the therapeutic agent can also act as a targeting agent and a therapeutic agent or possess one or more therapeutic properties.

Conformational Change.

In an aspect, the conjugation of one or more therapeutic agents (e.g., rituximab, blinatumomab, trastuzumab, pertuzumab, cetuximab, panitumumab) to a nanomaterial (e.g., graphene oxide) can involve non-covalent conjugation. Such non-covalent conjugation can be attributed to the conversion of a cytostatic therapeutic agent (e.g., one that inhibits cell growth) to a cytotoxic therapeutic agent (e.g., one that kills cells). The killing of cells can be one of a number of mechanisms, including apoptosis and non-apoptotic mechanisms. Accordingly, the nanomaterial complexes described herein can kill the malignant cells (e.g., lymphoma, cancer) without requiring the use of other cytotoxic medicines such as doxorubicin or other cytotoxic chemotherapeutics. Carrying out the methods of treatment as described above, it is possible to kill cancer cells while minimizing cytotoxic side effects that can lead to or are associated with life-threatening infections. Further, by converting a cytostatic therapeutic agent to a cytotoxic therapeutic agent that is also cell specific can have the additional advantage of preserving the immune system.

In some aspects, the conformational change in the association of the therapeutic agent and nanomaterial can be irreversible. Even in such cases, the therapeutic agent remains active and capable of binding to its target and producing an effect.

The methods disclosed herein related to the process of producing the compositions comprising one or more therapeutic agents non-covalently conjugated to a nanomaterial and nanomaterial complexes as described herein can be readily modified to produce a pharmaceutically acceptable dispersion. Pharmaceutical compositions including such dispersions and methods of administering them are accordingly within the scope of the present disclosure.

Pharmaceutical Compositions

As disclosed herein, are pharmaceutical compositions, comprising the composition described herein (e.g., one or more therapeutic agents non-covalently conjugated to a nanomaterial) and a pharmaceutical acceptable carrier. In some aspects, the therapeutic agent is an anti-cancer agent such as an anti-CD20 monoclonal antibody and the pharmaceutical composition is formulated for intravenous administration. In some aspects, the therapeutic agent is an anti-cancer agent such as an anti-HER1 or an anti-HER2 monoclonal antibody and the pharmaceutical composition is formulated for intravenous administration. In some aspects, the therapeutic agent is an anti-cancer agent such as an anti-CD19 monoclonal antibody and the pharmaceutical composition is formulated for intravenous administration. In some aspects, the therapeutic agent is an immunosuppressant agent such as an anti-CD3 monoclonal antibody and the pharmaceutical composition is formulated for intravenous administration. In some aspects, the therapeutic agent is an immunosuppressant agent such as an anti-T antibody and the pharmaceutical composition is formulated for intravenous administration. In some aspects, the compositions disclosed herein can further comprise an immunomodulator. In some aspects, the compositions disclosed herein can be administered alone or in combination with an immunomodulator also associated with the nanomaterial. In such aspects, the compositions disclosed herein and the immunomodulator can be administered as one or more pharmaceutical compositions, and if separately, can be administered simultaneously or sequentially in any order. The compositions of the present disclosure can also contain a therapeutically effective amount of a composition comprising one or more therapeutic agents non-covalently conjugated to a nanomaterial as described herein. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. In an aspect, the compositions described herein can be formulated for an intratumor injection or injections into body cavities including but not limited to the peritoneal cavity or pleural cavity or any other cavity or region of the body that is involved or contains a tumor. Aerosol inhalation can also be used to deliver the compositions described herein. Thus, compositions can be prepared for parenteral administration that includes the therapeutic agent conjugated to a nanomaterial dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

Methods of Treatment

Disclosed herein, are methods of treating a patient with cancer, the method comprising: (a) identifying a patient in need of treatment; and (b) administering to the patient a therapeutically effective amount of the pharmaceutical composition comprising a therapeutic agent non-covalently bound to a nanomaterial, and (c) a pharmaceutically acceptable carrier.

The pharmaceutical compositions described above can be formulated to include a therapeutically effective amount of a composition comprising a therapeutic agent non-covalently bound to a nanomaterial (e.g., graphene oxide). Therapeutic administration can encompass prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cancer, autoimmune disease or disorder or rejection of transplanted organ, tissue or cell.

In an aspect, the pharmaceutical compositions disclosed herein comprise one or more therapeutic agents wherein the therapeutic agent is an anti-CD20 monoclonal antibody. Examples of anti-CD20 monoclonal antibodies include but not limited to ofatumumab, rituximab, tositumomab, obinutuzumab, ibritumomab or a biologically active variant thereof. In an aspect, the anti-CD20 monoclonal antibody is rituximab. In some aspects, the pharmaceutical compositions can further comprise a therapeutically effective amount of an immunomodulator.

In an aspect, the pharmaceutical compositions disclosed herein comprise one or more therapeutic agents wherein the therapeutic agent is an anti-CD19 monoclonal antibody. Examples of anti-CD19 monoclonal antibodies include but not limited to blinatumomab (also referred to as Blincyto® and AMG103), GBR 401, coltuximab ravtansine (also known as SAR3419), denintuzumab mafodotin (also known as SGN-CD19A and SGN-19A) and taplitu-momabpaptox or a biologically active variant thereof. In an aspect, the anti-CD19 monoclonal antibody is blinatumomab. In some aspects, the pharmaceutical compositions can further comprise a therapeutically effective amount of an immunomodulator.

In an aspect, the pharmaceutical compositions disclosed herein comprise one or more therapeutic agents wherein the therapeutic agent is an anti-HER1 monoclonal antibody. Examples of anti-HER1 monoclonal antibodies include but are not limited to cetuximab (also referred to as Erbitux®), and panitumumab (also referred to as ABX-EGF, Vectibix®) or a biologically active variant thereof. In an aspect, the anti-HER1 monoclonal antibody is cetuximab and/or panitumumab. In some aspects, the pharmaceutical compositions can further comprise a therapeutically effective amount of an immunomodulator.

In an aspect, the pharmaceutical compositions disclosed herein comprise one or more therapeutic agents wherein the therapeutic agent is an anti-HER2 monoclonal antibody. Examples of anti-HER2 monoclonal antibodies include but are not limited to trastuzumab (also referred to as Herclon® and Herceptin®) and pertuzumab (also referred to as 2C4 and Perjeta®) or a biologically active variant thereof. In an aspect, the anti-HER2 monoclonal antibody is trastuzumab and/or pertuzumab. In some aspects, the pharmaceutical compositions can further comprise a therapeutically effective amount of an immunomodulator.

In an aspect, the pharmaceutical compositions disclosed herein comprise one or more therapeutic agents wherein the therapeutic agent is an anti-CD3 monoclonal antibody. Examples of anti-CD3 monoclonal antibodies include but are not limited to muromonab-CD3 (also referred to as Orthoclone OKT3®), otelixizumab (also referred to as TRX4), teplizumab (also referred to as MGA031 and hOKT3γ1), and visilzumab (also referred to as nuvion) or a biologically active variant thereof. In an aspect, the anti-CD3 monoclonal antibody is muromonab-CD3, otelixizumab, teplizumab and/or visilzumab. In some aspects, the pharmaceutical compositions can further comprise a therapeutically effective amount of an immunomodulator.

In an aspect, the pharmaceutical compositions disclosed herein comprise one or more therapeutic agents wherein the therapeutic agent is a horse- or rabbit-derived antibody. In an aspect, the horse- or rabbit-derived antibody is against human T cells. An example of a horse- or rabbit-derived antibody is anti-thymocyte globulin or a biologically active variant thereof. In an aspect, the horse- or rabbit-derived antibody is anti-thymocyte globulin. In some aspects, the pharmaceutical compositions can further comprise a therapeutically effective amount of an immunomodulator.

The pharmaceutical compositions described herein can be administered to the subject (e.g., a human patient) in an amount sufficient to delay, reduce, or preferably prevent the onset of clinical disease. Accordingly, in some aspects, the patient is a human patient. In therapeutic applications, compositions are administered to a subject (e.g., a human patient) already with or diagnosed with cancer in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effect amount includes amounts that provide a treatment in which the onset or progression of the cancer is delayed, hindered, or prevented, or the cancer or a symptom of the cancer is ameliorated. One or more of the symptoms can be less severe. Recovery can be accelerated in an individual who has been treated.

In some aspects, the cancer is a primary, secondary, refractory or relapsing tumor. In other aspects, the primary, secondary, refractory or relapsing tumor is a blood cell tumor. In another aspect, the cancer is associated with the expression of a B lymphocyte associated antigen (e.g., CD20, CD19). In an aspect, the primary, secondary, refractory or relapsing tumor is a lymphoma. In an aspect, the primary, secondary, refractory or relapsing tumor is a sarcoma. The sarcoma can be osteosarcoma. In an aspect, the cancer is associated with the expression (or overexpression) of HER2. In another aspect, the primary, secondary, refractory or relapsing tumor is a carcinoma. The carcinoma can be pancreatic carcinoma. In an aspect, the pancreatic carcinoma can be pancreatic adenocarcinoma or pancreatic ductal carcinoma. The carcinoma can also be lung, colon, gastroesophageal, head or neck cancer. In an aspect the cancer is associated with the expression (or overexpression) of HER1.

Disclosed herein, are methods of treating a patient with cancer. The cancer can be any cancer. In some aspects, the cancer affects the blood, bone marrow, lymph or lymphatic system. Tumors of the blood can also affect the circulatory system and/or the immune system. Blood cancers can also be referred to as liquid cancers and can affect red blood cells, white blood cells or a combination of both. Cancers of the blood can also be called hematological malignancies and can be further classified as leukemias, lymphomas and myelomas. Examples of leukemias include acute lymphoblastic leukemia, acute myelogenous leukemia, chronic lymphocytic leukemia, chronic myelogenous leukemia and acute monocytic leukemia. Examples of lymphomas include Hodgkin's lymphoma and non-Hodgkins lymphomas. Classical Hodgkin lymphoma can be further classified into the following subtypes: nodular sclerosing Hodgkin lymphoma, mixed-cellularity, lymphocyte-rich or lymphocytic predominance and lymphocyte depleted. Nodular lymphocyte predominant Hodgkin's lymphoma may express CD20. Non-Hodgkin lymphomas include all subtypes of lymphoma except Hodgkin's lymphoma. Hodgkin lymphoma cells express CD30. B cell non-Hodgkin lymphomas express CD20. Examples of B cell non-Hodgkin lymphomas include B-cell chronic lymphocytic leukemia/small lymphocytic lymphoma. B-cell prolymphocytic leukemia, lymphoplasmacytic lymphoma, splenic marginal zone B-cell lymphoma (±villous lymphocytes), hairy cell leukemia, plasma cell myeloma/plasmacytoma extranodal marginal zone B-cell lymphoma of the MALT type, nodal marginal zone B-cell lymphoma (±monocytoid B cells), follicular lymphoma, mantle cell lymphoma, marginal zone cell lymphoma, diffuse large B-cell lymphomas (including mediastinal large B-cell lymphoma and primary effusion lymphoma) and Burkitt lymphoma. In an aspect, the blood cell tumor is lymphoma. In an aspect, the lymphoma is a non-Hodgkin's lymphoma.

The methods disclosed herein can also be applied to other diseases or disorders. In an aspect the methods described herein can be used to treat autoimmune disorders (e.g., rheumatoid arthritis, systemic lupus erythematosis, myasthenia gravis, and multiple sclerosis); or to prevent graft rejection (i.e., minimize the immune system response to transplantation of one or more tissues, organs or cells) and/or treat or prevent the signs and symptoms associated with graft rejection.

Disclosed herein, are methods of treating a patient to prevent or minimize or reduce transplant (e.g., graft) rejection (e.g., acute rejection). In an aspect, the transplant can be an allogeneic renal, heart or liver transplant. In an aspect the transplant can be protected or the signs and symptoms associated with graft rejection can be reduced by targeting CD3 receptors present on T cells. Binding of a therapeutic agent (e.g., antibody) to the CD3 receptor expressed on the T cell can block T cell activities and/or prevent apoptosis of the T cells, thus, leading to protection of the transplant. In an aspect, CD3 receptor antibodies include but are not limited to muromonab-CD3, otelixizumab, teplizumab and visilzumab. In an aspect, the transplant can be protected or the signs and symptoms associated with graft rejection can be further reduced by administering anti-thymocyte globulin (e.g., horse- or rabbit-derived antibodies against human T cells).

The methods of treatment disclosed herein can also include a step of killing a specific cell type (e.g., a B cell expressing CD20, a B cell expressing CD19, a cell expressing HER1, or a cell expressing HER2). The methods of treatment can further include a step of contacting a cell with the compositions and/or nanocomplexes described herein.

In an aspect, the methods of treatment disclosed herein can also include the administration of a therapeutically effective amount of radiation therapy, immunotherapy, chemotherapy, stem cell transplantation or a combination thereof.

Therapeutically effective amounts can be determined empirically by one skilled in the art. Single or multiple administrations of the pharmaceutical compositions disclosed herein can be carried out with dosage levels and the timing pattern determined by the treating physician. Amounts effective for the uses described above can depend on the severity of the cancer (or other disease or other condition such as graft transplantation) and the weight and general state and health of the subject, but generally range from about, for example, for rituximab, 0.0375-375 mg/m², of an equivalent amount of the composition comprising a therapeutic agent conjugated to a nanomaterial per dose per subject. Suitable regimes for initial administration and booster administrations are typified by an initial administration followed by repeated doses at one or more hourly, daily, weekly, or monthly intervals by a subsequent administration. For example, a subject can receive a therapeutic agent (e.g., rituximab) conjugated to a nanomaterial in the range of about 0.0375-375 mg/m² or equivalent dose as compared to unbound or free therapeutic agent(s) per dose one or more times per week (e.g., 2, 3, 4, 5, 6, or 7 or more times per week) for four to 8 weeks. For example, a subject may receive a dose of rituximab ranging from about 0.0375-375 mg/m² (e.g., 375 mg/m², 37.5 mg/m², 3.75 mg/m², 0.375 mg/m², 0.0375 mg/m²) dose per week. A subject can also receive a therapeutic agent (e.g., rituximab) conjugated to a nanomaterial in the range of 0.0375-375 mg/m² per dose once every two or three weeks. An effective dose of trastuzumab can range from about, for example, 2 mg/kg to 6 mg/kg, of an equivalent amount of the composition comprising a trastuzumab conjugated to a nanomaterial per dose per subject. A subject may receive a weekly dose of trastuzumab conjugated to a nanomaterial of about 4 mg/kg, generally administered intravenously over a 90 minute period of time the first week of treatment, followed by a dose of 2 mg/kg over 30 minutes every week thereafter for 12 to 18 weeks. One week following the last weekly dose of trastuzumab, 6 mg/kg can be infused over 30 to 90 minutes every week for three weeks. An effective dose of pertuzumab can range from about, for example, 420 mg to 840 mg, of an equivalent amount of the composition comprising a pertuzumab conjugated to a nanomaterial per dose per subject. For example, a subject may receive an initial dose of 840 mg infused intravenously over a 60 minute time period, followed by 420 mg infused intravenously over to 60 minutes every three weeks. An effective dose of cetuximab can range from about, for example, 250 mg/m² to 400 mg/m², of an equivalent amount of the composition comprising cetuximab conjugated to a nanomaterial. A subject may receive an initial dose of 400 mg/m² of cetuximab infused intravenously over a 2 hour time period, followed by 250 mg/m² of cetuximab infused intravenously weekly thereafter. An effective dose of panitumumab can range from about for example, 6 mg/kg to 1 g/kg, of an equivalent amount of the composition comprising panitumumab conjugated to a nanomaterial. A subject, for example, can receive about 6 mg/kg to 1 g/kg infused intravenously over 60 to 90 minutes every two weeks.

The therapeutically effective dose of the disclosed compositions can be administered using any medically acceptable mode of administration. One of ordinary skill in the art can contemplate any of the modes of administration known. For example, the compositions described herein can be administered according to the recommended mode of administration listed on the package insert of a commercially available agent. The dose of a composition described herein can comprise 0.01 mg to about 2.5 g/kg/day or equivalent dose. The dosage of a compositions described herein can be measured in any appropriate unit including but not limited to mg/kg, g/kg, mCi, mg/m² or at a fixed dose.

For example, a subject can receive a composition as described herein in the range of about 0.3 mg to 2,000 mg or equivalent dose as compared to unbound or free therapeutic agent(s) per dose one or more times per week (e.g., 2, 3, 4, 5, 6, or 7 or more times per week) for four to 8 weeks or longer. The treatment regimen can be carried out in multiple steps or in cycles, and/or according to the attending physician and/or the package insert of a commercially available therapeutic agent.

The total effective amount of a therapeutic agent conjugated to a nanomaterial in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month; sometimes referred to as cycle). Alternatively, continuous intravenous infusions sufficient to maintain therapeutically effective concentrations in the blood are also within the scope of the present disclosure.

The therapeutically effective amount of one or more of the therapeutic agents present within the compositions described herein and used in the methods as disclosed herein applied to mammals (e.g., humans) can be determined by one of ordinary skill in the art with consideration of individual differences in age, weight, and other general conditions (as mentioned above). Because the therapeutic agent(s) non-covalently conjugated to a nanomaterial of the present disclosure can be stable in serum and the bloodstream and in some cases more specific, the dosage of these compositions including any individual component can be lower (or higher) than an effective dose of any of the individual components when unbound (i.e., free). Accordingly, in some aspects, the therapeutic agent administered has increased efficacy, increased avidity or reduced side effects when administered non-covalently conjugated to a nanomaterial as compared to when the therapeutic agent (e.g., anti-cancer agent) is administered alone or not conjugated to a nanomaterial.

Kits

The kits can include a composition comprising a nanomaterial (e.g., graphene oxide) and one or more therapeutic agents, wherein the nanomaterial (e.g., graphene oxide) is non-covalently bound to the one or more therapeutic agents; and suitable instructions (e.g., written and/or audio-, visual-, or audiovisual material). In an aspect, the composition is in the presence of a low salt solution. The composition can further comprise a pharmaceutically acceptable carrier. In an aspect, the kit includes a pharmaceutical composition as described herein that is packaged together with instructions for use. The kits can also include one or more of the following: diluents, sterile fluid, syringes, a sterile container, gloves, vials or other containers, pipettes, needles and the like.

### EXAMPLES

### Example 1: Rituximab can be Stably Loaded onto Graphene Oxide

Graphene oxide (GO) consists of sp2-hybridized carbon rings with hydroxyl and carboxyl groups and thus has the potential to interact with antibody molecules through pi-stacking and hydrophobic interactions as well as hydrogen and ionic bonds.

GO dispersion (Sigma) was sonicated with a probe sonic dismembrator (Fisher Scientific; Model 550) for 120 minutes at amplitude 3.5 and filtered through a 0.2 μm filter. To non-covalently conjugate rituximab (RTX) with GO, 0.2 mg sonicated GO and 1 mg RTX were mixed in 1 ml 10% PBS, PBS, or water, and incubated at 37° C. for one to 12 hours under constant agitation. The incubated non-covalently conjugated mixture, designated RTX/GO, was used for staining, cell culture or in vivo experiments. To determine the amount of RTX non-covalently bound to GO, GO was precipitated from the mixture by centrifugation, boiled in dithiothreitol-containing loading buffer and subjected to 12%0/polyacrylamide gel electrophoresis (PAGE).

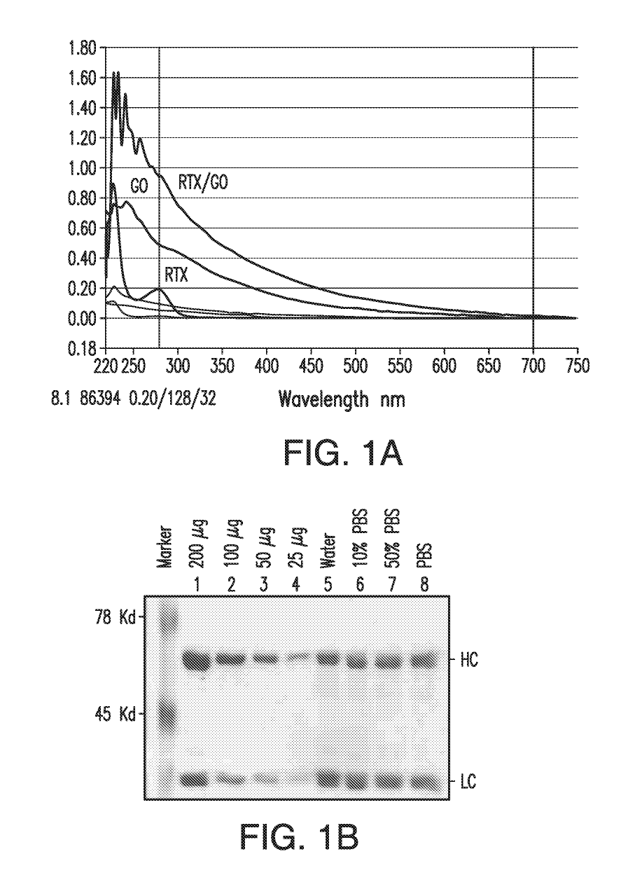

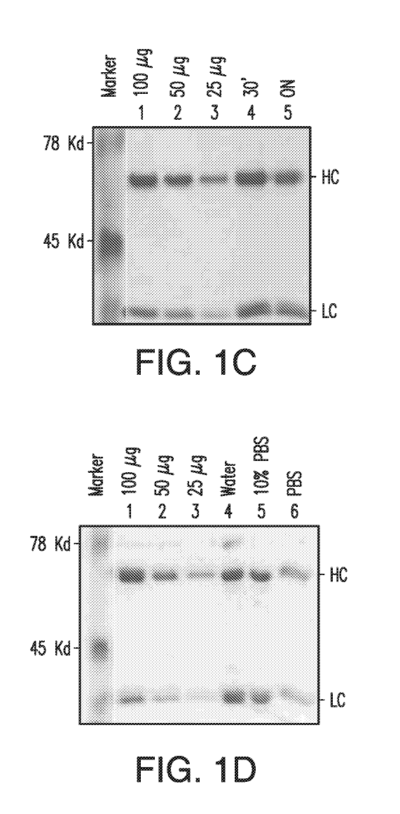

To determine whether RTX can be effectively loaded onto GO through non-covalent interactions, RTX was incubated with GO in buffered saline at low salt concentration (10% PBS at 37° C.) overnight. RTX/GO interactions were monitored using UV-Vis spectra. Free RTX absorption peaked at 280 nm whereas free GO absorbed across a broad spectrum, peaking at 230 nm (FIG. 1A), as previously reported (Huang, P., et al. Theranostics, 2011, 1: p. 240-50). The mixture of RTX and GO (e.g., RTX/GO) gave rise to an absorption spectrum similar to that of free GO but with substantially enhanced magnitude, suggesting RTX is non-covalently conjugated to GO.

To examine the stability of RTX/GO and determine the amount of RTX stably and non-covalently conjugated to GO, GO was isolated from the RTX/GO dispersion by centrifugation, thoroughly washed at room temperature with water, 10%, 50% and normal PBS, and examined by SDS page for the amount of RTX non-covalently conjugated to GO. Compared to the known amount of RTX loaded in parallel in the gel, the amount of RTX non-covalently conjugated to GO can be estimated. Approximately 100 μg RTX remained non-covalently bound to 20 μg of GO (ratio of 5 to 1) after the GO underwent washing under all of these conditions (FIG. 1B). Washing was also carried out with normal PBS at 37° C. for 30 minutes or overnight (18 hours) and a substantial (about 80%) amount of RTX remained non-covalently bound to GO even after overnight washing (FIG. 1C). When undiluted (normal 0.9% NaCl) PBS was used as incubation buffer, there was a significant (about 70%) reduction in the amount of RTX stably and non-covalently bound to GO as compared to when water or 10% PBS was used as an incubation buffer (FIG. 1D).

These results demonstrate that the overnight incubation of RTX with GO in low salt concentrations at 37° C. results in stable, non-covalently conjugated RTX to GO whereas physiological salt concentrations do not support stable non-covalent conjugation of RTX to GO. Despite that normal salt concentrations disfavor stable binding between RTX and GO, it can no longer dissociate RTX from GO that is already non-covalently bound to GO under the low salt conditions (water or 10% PBS). These findings suggest that the non-covalently binding of RTX to GO under low salt condition induced conformational changes in GO. These conformational changes are not reversible even in normal salt concentrations, and thus prevent RTX/GO dissociation. The stable non-covalent binding between RTX and GO implicates that stability of RTX/GO in vivo where the salt concentration is normal (0.9% NaCl).

### Example 2: GO-Non-Covalently Bound to RTX Remains Reactive with CD20 and Induces CD20 Capping

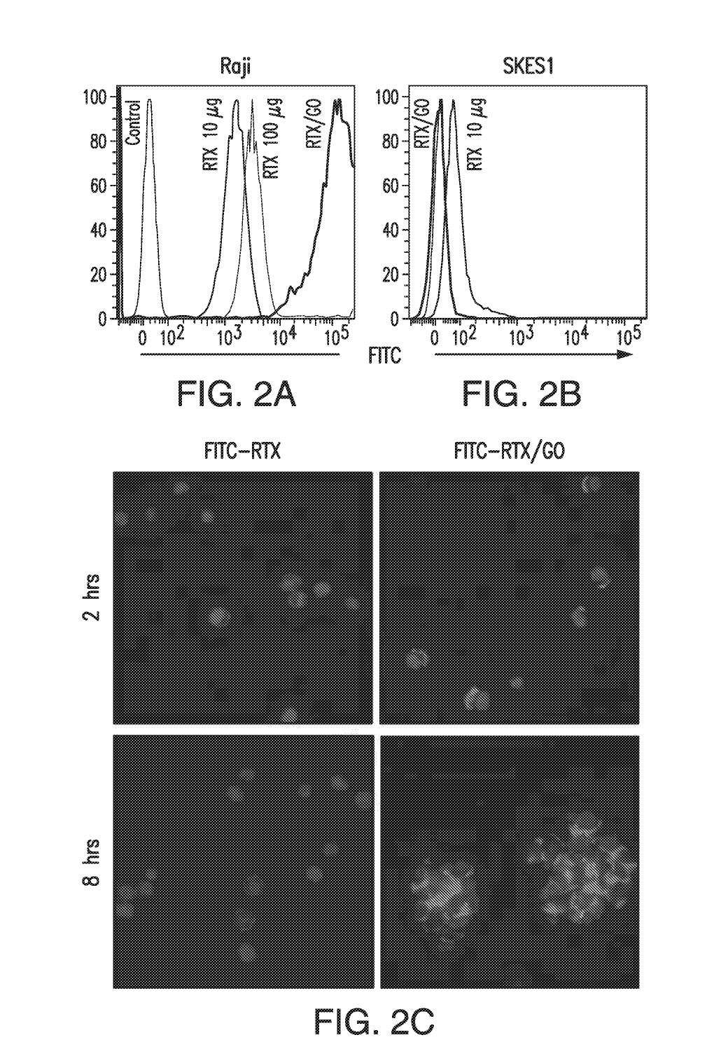

To test whether the stochastic non-covalent binding of RTX to GO during the loading reaction blocks the antigen-binding sites of RTX, such that GO/RTX is no longer reactive with CD20, the following set of experiments were carried out. To examine this possibility, FITC-conjugated RTX and GO were mixed at a 5 to 1 weight ratio (e.g., 5 μg of RTX and 1 μg of GO) in 10% PBS and incubated overnight. The non-covalent mixture (designated RTX/GO) was used to stain Raji cells, a CD20-positive Burkitt's lymphoma cell line for flow cytometry analysis (FIG. 2A). Free FITX-RTX was used as a control. GO/RTX-FITC not only stained Raji cells positively but also gave rise to much brighter (about 100 times) staining as compared to free RTX-FITC (FIG. 2A). The staining by RTX/GO was CD20 specific because RTX/GO did not stain a CD20^{neg} Ewing sarcoma cell line, SKES1 (FIG. 2B).

Given that the affinity of RTX is the same, the substantially enhanced CD20 staining by RTX-FITC/GO suggests that the RTX non-covalently conjugated to GO has higher avidity compared to free RTX. As avidity is a function of accumulated strength of multiple affinities of multivalent antibodies, the enhanced binding capacity of FITC-RTX/GO suggests that RTX non-covalently conjugated to GO is multivalent. To assess the multivalency, the stained cells were examined by immunofluorescence microscopy. Two hours after staining, uniform membranous fluorescence was visualized on Raji cells stained with free RTX-FITC (FIG. 2C). In contrast, coarse fluorescent aggregates were identified at one pole of the cells on the GO/RTX-FITC stained Raji cells, which is consistent with CD20 capping (FIG. 2C). Eight hours post staining, GO/RTX-FITC stained Raji cells were brought together forming clumps, consistent with homotypic aggregation (FIG. 2C), whereas free RTX-FITC stained cells remained as single cells with uniform membranous fluorescence (FIG. 2C). No intracellular fluorescence was detected in the stained cells even 16 hours post staining (not shown), indicating that the CD20 caps were not endocytosed. Since capping is the result of crosslinking of the ligands by multivalent antibodies, this result indicates that the RTX/GO is multivalent.

### Example 3: RTX Non-Covalently Conjugated to GO is Cytotoxic to Malignant B Cells

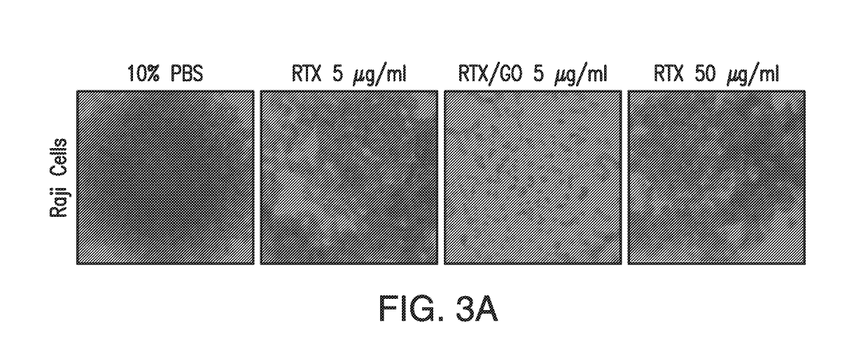

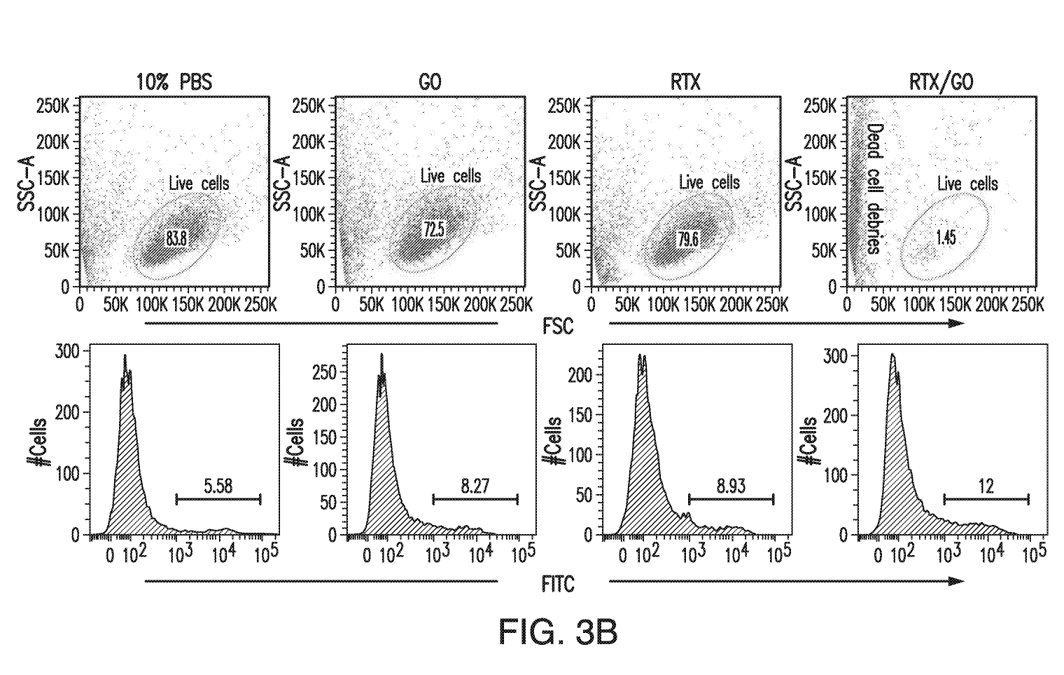

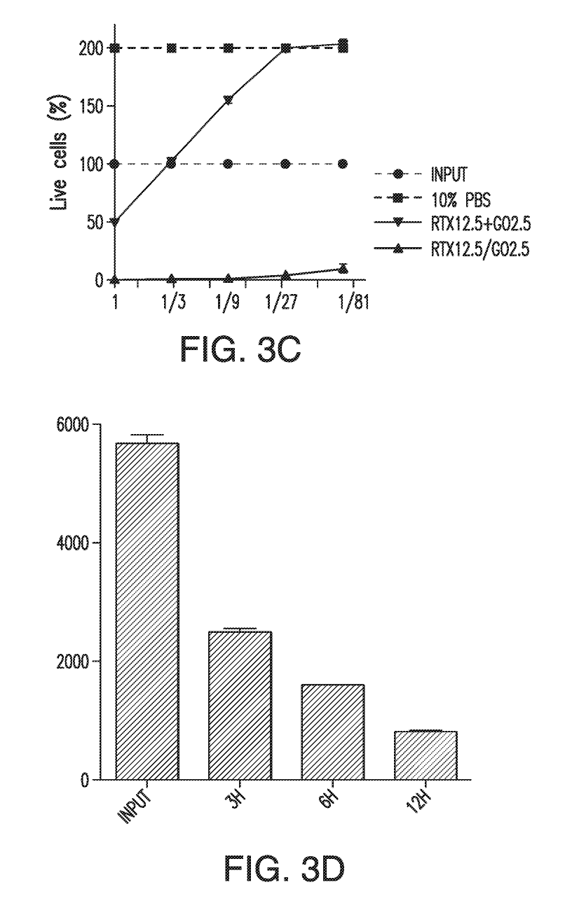

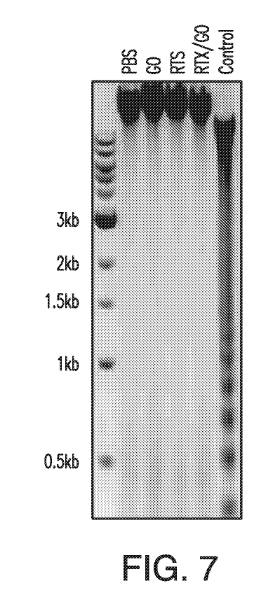

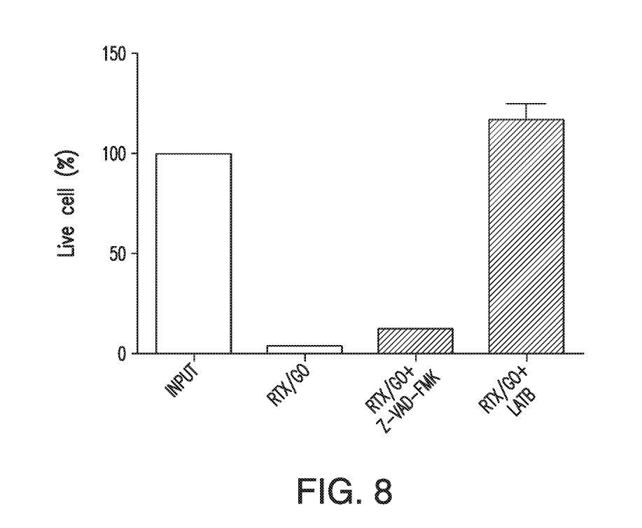

Given the previous reports that crosslinking CD20 causes B cell death, the finding of CD20 capping by RTX/GO (see above) raised the possibility that RTX/GO might be cytotoxic to CD20+ cells. To this end, Raji cells were cultured overnight with RTX/GO at 5 μg/ml, PBS, free RTX at 5 or 50 μg/ml, or GO as controls. The cells cultured with free RTX at 5 or 50 μg/ml underwent robust proliferation, though at a slightly slower rate compared to the PBS controls, whereas the cells cultured with RTX/GO showed no evidence of proliferation on microscopic examination (FIG. 3A). Flow cytometry showed that the cultures with RTX/GO contained primarily necrotic cell debris, with only rare viable cells (FIG. 2B). RTX/GO was further tested in Raji cell culture, and showed almost complete loss of live Raji cells, as determined by the LIVE/DEAD cell stain or SSC on flow cytometry (FIG. 3B, upper panel), indicating that RTX/GO kills Raji cells. In contrast, Raji cells in culture with PBS, RTX or GO continued to proliferate (FIG. 3D). To examine the nature of the cell death, the cultures were stained with the apoptotic marker Annexin V (FIG. 3B, lower panel). As expected, most cells cultured with PBS, GO, and RTX were viable and Annexin V negative. Unexpectedly, however, was the finding that a small percentage (12%) of cells in the RTX/GO culture were Annexin V positive despite the fact that the culture contained primarily necrotic cell debris. DNA electrophoresis revealed no apoptotic DNA fragmentation (FIG. 7). Apoptosis positive control DNA was isolated from mouse thymocytes that were cultured in plane medium for two days. These results indicated that cell death induced by RTX/GO was not through apoptosis. In line with this, the pan-caspase inhibitor Z-VAD-FMK did not significantly alleviate RTX/GO-induced Raji cell death (FIG. 8), confirming a caspase independent mechanism for the cell death. As capping of surface molecules requires reorganization of the actin network, an actin polymerization inhibitor, latrunculin B (LatB), was tested for its effect on RTX/GO-mediated cytotoxicity. LatB completely abrogated RTX/GO-induced Raji cell death (FIG. 8), indicating that the cell death involves an actin-dependent mechanism. Taken together, these results demonstrate that RTX/GO is cytotoxic to Raji cells. Free RTX is only mildly inhibitory, even at high concentrations, consistent with previous reports (Li, B., et al. Blood, 2009. 114(24): p. 5007-15).

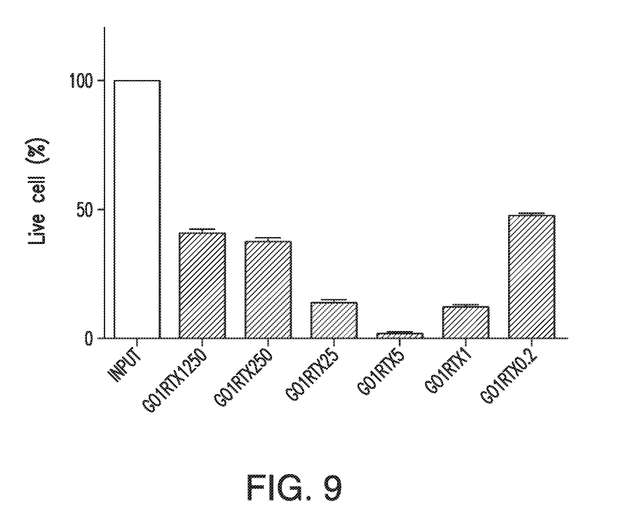

To examine the potency of RTX/GO, Raji cells were culture with decreasing concentrations of RTX/GO, starting at 12.5 μg/ml. RTX/GO eliminated Raji cells effectively even at very low concentrations (0.195 μg/ml). GO by itself exhibited an inhibitory effect on Raji cell proliferation when present at high concentrations, consistent with the previous reports that GO can cause dose-dependent oxidative stress in cells and induce a slight loss of cell viability at high concentrations (Seabra, A. B., et al. Chemical research in toxicology, 2014. 27(2): p. 159-68). The potent cytotoxicity of RTX/GO, however, was not directly related to the inhibitory/toxic effect of GO, because at low concentrations (0.078 μg/ml and lower) when GO is no longer inhibitory, RTX/GO at similar concentrations (0.25/0.078 μg/ml and lower) remained extremely cytotoxic and killed Raji cells (FIG. 3C). Adding high concentrations of free RTX (>12.5 μg/ml and 2.5 μg/ml, respectively) along with free GO to the culture at the time of culture set-up also induced Raji cell death to a mild degree. This is most likely results from low levels of RTX/GO formed by the presence of high concentrations RTX and GO in the culture. At low concentrations (0.25 and 0.078 μg/ml or lower), however, the onsite addition of free RTX and free GO showed no cytotoxicity or inhibitory effect (FIG. 3C). The lack of cytotoxic effect of the onsite addition of low concentrations of free RTX and free GO indicates that the mere simultaneous presence of RTX and GO in the culture is insufficient to generate cytotoxicity, but rather association of RTX with GO (e.g., non-covalent conjugation of RTX to GO) is required for Raji cell killing. To further investigate, a fixed concentration of GO was mixed with different concentrations of RTX generating RTX/GO with different RTX to GO ratios for Raji cell culture. All of the RTX/GO mixtures killed Raji cells to various degrees, but the most efficient killing occurred at a RTX to GO mass ratio of 5 to 1 (FIG. 9), corresponding to the GO binding capacity of RTX described above (FIG. 1). RTX at high concentrations diminished the killing capacity of RTX/GO, most likely because the presence of excessive free RTX was competing with RTX/GO to bind to CD20. These results further indicate that the potent cytotoxicity of RTX/GO results from RTX/GO-mediated CD20 crosslinking.

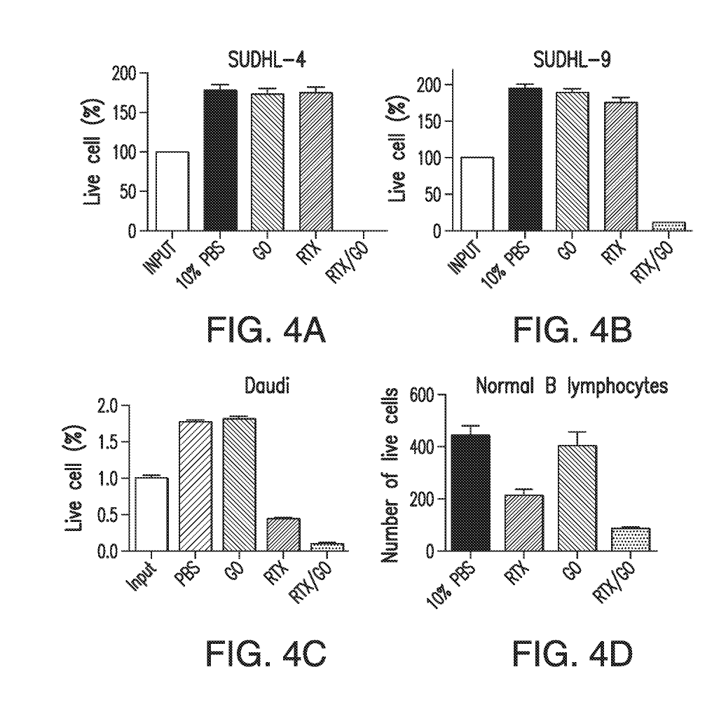

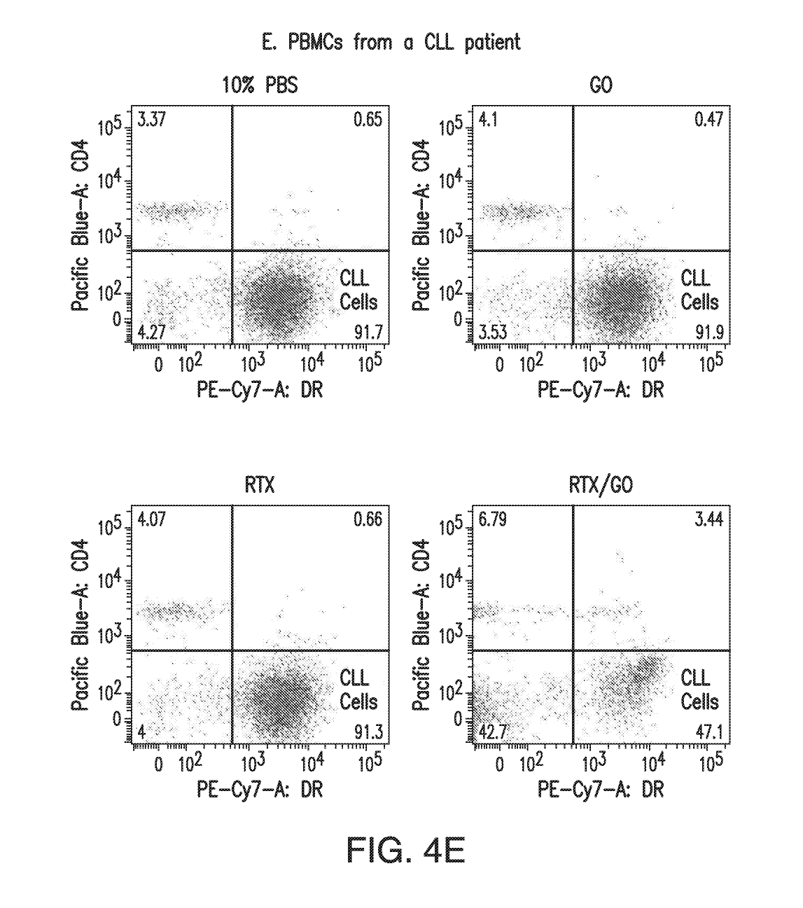

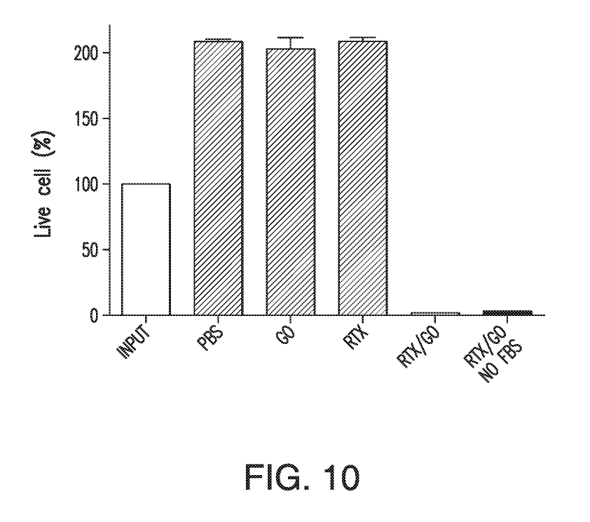

The highly potent cytotoxicity of RTX/GO implicates a fast killing process. To examine the speed of killing, the number of remaining viable Raji cells in culture with RTX/GO was followed at various time points in culture. Rapid loss of viable cells was noticed as early as 3 hours post culture. By 6 hours, the majority of Raji cells were already killed and became cell debris, with continuing loss of viable cells over time (FIG. 3D), demonstrating that RTX/GO kills at extremely fast rate. The fetal-calf serum used to supplement the culture media was heat-inactivated and therefore should not contain complement activity. To completely exclude the role of complements in the cell death, serum-free medium was used in culture with RTX/GO. RTX/GO killed Raji cells in the absence of serum with similar potency (FIG. 10). RTX/GO/ was also tested on two other lymphoma cell lines, SUDHL-4 and HUDHL-9, which are derived from high grade diffuse large B cell lymphoma. DLBCL. SUDHL. SUDHL-9, Daudi, normal B lymphocytes and PBMCs from a CLL patient were cultured for one day with 10% PBS, GO, RTX or RTX/GO. Consistent with previous reports that SUDHL-4 is sensitive to RTX (Kobayashi, H., et al. Cancer Med. 2013. 2(2): p. 130-43), RTX substantially inhibited SUDHL-4 proliferation even at low concentration (FIG. 4A). Nevertheless, no significant killing of SUDHL-4 cells was obtained with RTX even at high concentrations (up to 200 μg/ml). RTX/GO, however, continued to kill SUDHL-4 cells at very low concentrations. SUDHL-9 cells were less sensitive to RTX inhibition but nevertheless were killed by RTX/GO even at very low concentrations (0.2 μg/ml) (FIG. 4B). RTX/GO was also tested on another Burkitt lymphoma cell line Daudi (FIG. 4C), normal B cells (FIG. 4D), and primary lymphoma cells from a patient with chronic lymphocytic leukemia (CLL; FIG. 4E), and the results showed that RTX/GO potently killed all the cell types tested. These results demonstrate that while free RTX is only inhibitory, RTX/GO is highly cytotoxic with the capacity to rapidly kill CD20-positive target cells at very low concentrations independent of complement or ADCC.

### Example 4: RTX/pGO and RTX/GO, but not Free RTX, Rapidly Eliminates Metastatic Burkitts Lymphoma in a Xenograft Mouse Model

To study the ability of RTX/GO and RTX/pGO to eliminate lymphoma in vivo, a xenotransplant Burkitts lymphoma mouse model was deployed using immunodeficient NODrag^{ko}γ^{ko} (NRG) mice. The NRG mice lack T, B and NK cells and also have defective macrophages (O'Brien, B. A., et al. Diabetes, 2002. 51(8): p. 2481-8), and are, therefore, deficient of the host effector cells that free RTX relies on to eliminate the targeted cells. Systemic lymphoma was established by intravenously (iv) transplanting Raji cells into NRG mice. As shown in a previous study, disseminated Burkitts lymphoma developed in this mouse model predominantly involves the extranodal sites, especially the liver (Chao, M. P., et al. Blood, 2011. 118(18): p. 4890-901). Pegylated GO (pGO) was synthesized and used to load RTX to generate RTX/pGO mixtures for iv injection. Eight days after Raji cell transplantation, the mice were treated daily with iv administration of either pGO, RTX, or RTX/pGO. After four treatments, the mice were sacrificed for pathological examination.

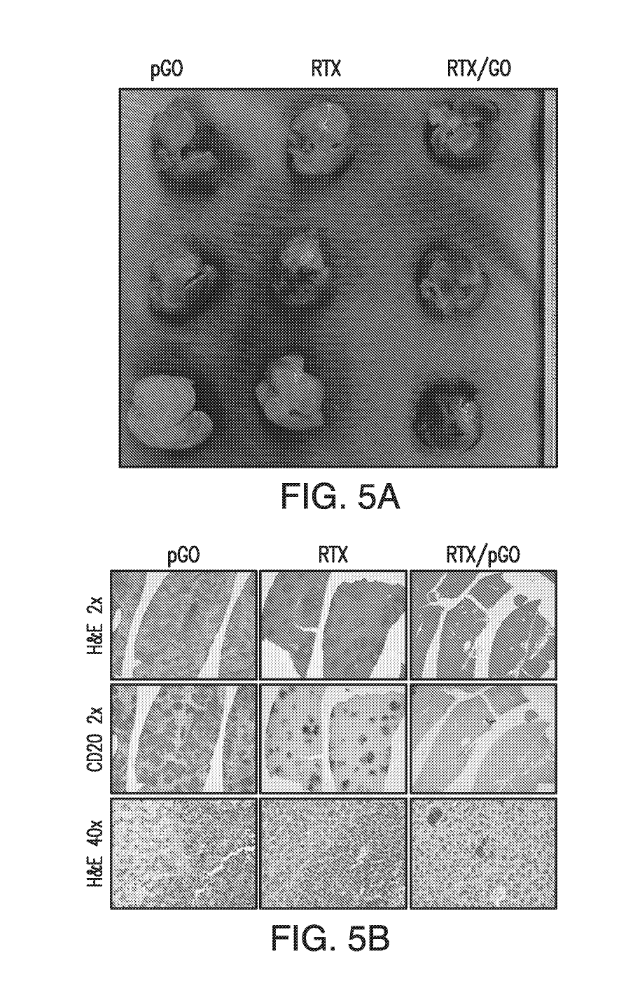

Marked hepatomegaly was identified in the livers of all the pGO as well as RTX-treated mice, though the organomegaly was milder in the RTX-treated group (FIG. 5A). The hepatomegaly was the result of disseminated lymphoma that was readily visible on the surfaces and sections of the livers on gross examination. In contrast, the livers of the RTX/pGO-treated mice appeared to be of normal size, completely free of gross lymphoma lesions. Consistent with gross examination, numerous lymphoma infiltrates were identified in the H&E stained liver sections of all the pGO- and free RTX-treated mice (FIG. 5B). Immunohistochemical stain for CD20 confirmed that the infiltrates consisted of CD20-positive cells, with smaller tumor burden in the liver of free RTX-treated mice as compared to the pGO-treated mice (FIG. 5). The reduction in tumor burden in the free RTX-treated mice may result from a combination of the inhibitory effect of RTX as well as RTX-induced complement-mediated cytotoxicity that eliminates some lymphoma cells. Despite the antitumor activity of free RTX, however, the lymphomas in the free RTX-treated mice appeared to continue growing, as brisk mitotic figures were present in the infiltrating lymphoma cells in the liver (FIG. 5B). In contrast, no evident lymphoma infiltrates were identified in the liver of pGO/RTX-treated mice on either H&E stained sections or by CD20-positive staining. Under high magnification of microscopic examination, rare degenerated cellular aggregates were identified, consistent with dead Raji cells.

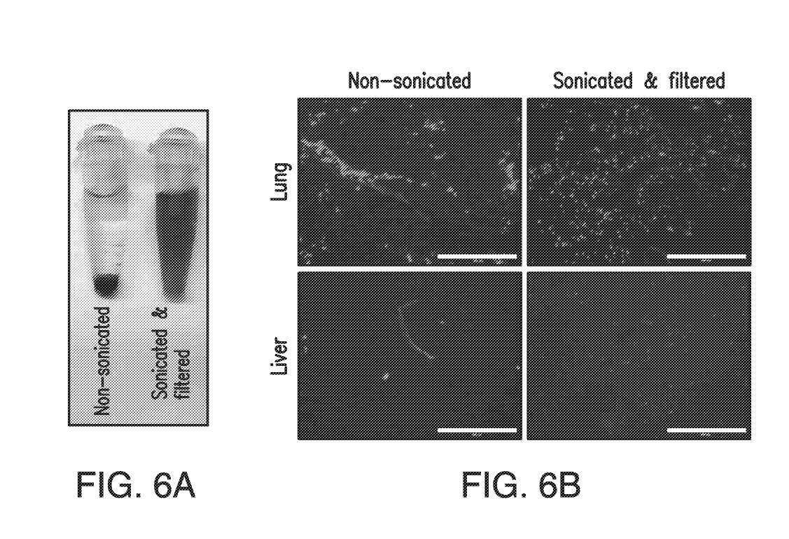

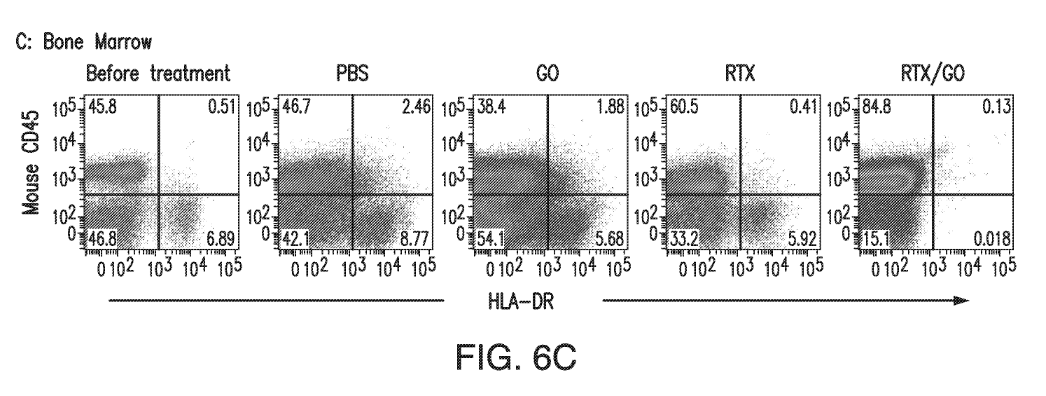

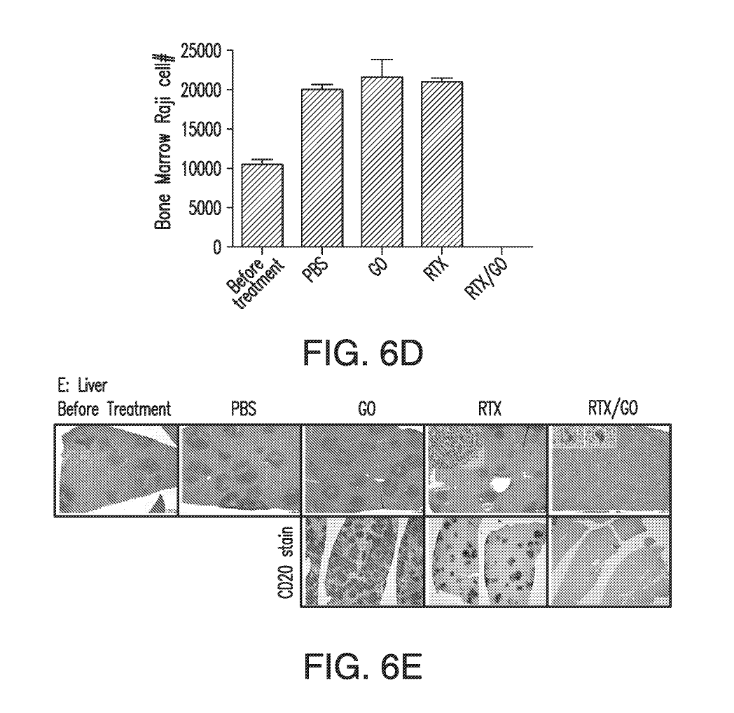

Additional experiments show the therapeutic effects of RTX/GO (without pegylation) in vivo. As other studies reported limited stability and biodistribution of GO due its size, GO used in this experiment was sonicated and filtered through a 0.22 μm filter. Before sonication, GO could not pass through 0.22 μm filters, but it readily passed through after sonication. Non-sonicated or sonicated and filtered GO was mixed with human serum (10 μg/ml). Non-sonicated GO precipitated within four hours while sonicated and filtered GO remained in dispersion indefinitely. Sonicated GO remained in dispersion indefinitely in serum while the non-sonicated GO precipitated within 4 hours (FIG. 6A). RTX/GO made of either non-sonicated or sonicated and filtered GO with RTX-FITC was injected into NRG mice iv and organs were sampled 2 hours after injection. After iv injection, RTX/GO made of non-sonicated GO was almost completely trapped within the vasculature of the lung, undetectable in the lung tissue or distant organs such as liver (FIG. 6B). In contrast, RTX/GO made of sonicated GO was almost completely detected in lung and liver tissue without intra-vascular retention. Eight days post Raji cell transplantation, lymphoma were readily identified in the bone marrow (FIGS. 6C-E), and the mice were then treated with iv PBS, GO, RTX, or RTX/GO every two to 3 days for 3 times. Raji cells are identified as mouse CD45^{neg} and human HLA-DR^{pos} cells. Pathological examination was performed 3 days after the last treatment. Extensive lymphoma was identified in the bone marrows and livers of all the PBS, GO and RTX-treated mice but none of the RTX/GO-treated mice (FIG. 6C-D).

These findings confirm the in vitro results showing that RTX/pGO or RTX/GO are directly cytotoxic to lymphoma cells, and also demonstrate that RTX/pGO or RTX/GO has a capacity to diffuse out of the blood circulation, penetrate through the tissue to reach target cells and rapidly eliminate established lymphomas in the absence of host effector mechanisms, while free RTX fails to do so.

### Example 5: Effects of Covalent and Non-Covalent Conjugation of a Therapeutic Agent to Graphene Oxide on Lymphoma Cells

The effects of the antibody RTX conjugated either covalently or non-covalently to graphene oxide were compared. As noted above, the Examples described herein demonstrate that non-covalently-bonded RTX/GO (e.g., RTX/GO) is cytotoxic to lymphoma cells in the absence of a drug or drugs classified as cytotoxic. Thus, based on the studies disclosed herein, RTX non-covalently conjugated to GO serves a dual role: guiding the delivery of a therapeutic agent and killing target cells (e.g., lymphoma cells). In a previously published study (Sun et al. (2008) Nano Res. 1(3): 203-212), doxorubicin (DOX) was non-covalently loaded onto a covalent construct of GO, PEG, and the antibody RTX (e.g., GO-PEG-Ab/DOX; Ab=RTX) that targets B cells. In this orientation, GO is used to non-covalently carry DOX, a cytotoxic drug, and covalently carry (through PEG) the antibody (RTX), In contrast and as described herein, GO can also be used as a scaffold for non-covalent association of multiple RTX molecules on individual GO molecules to generate RTX/GO. Below, GO covalently conjugated to RTX is compared to GO non-covalently conjugated to RTX. The results show that RTX/GO (i.e., RTX non-covalently conjugated to GO) has several advantages over the covalently bound GO to RTX.

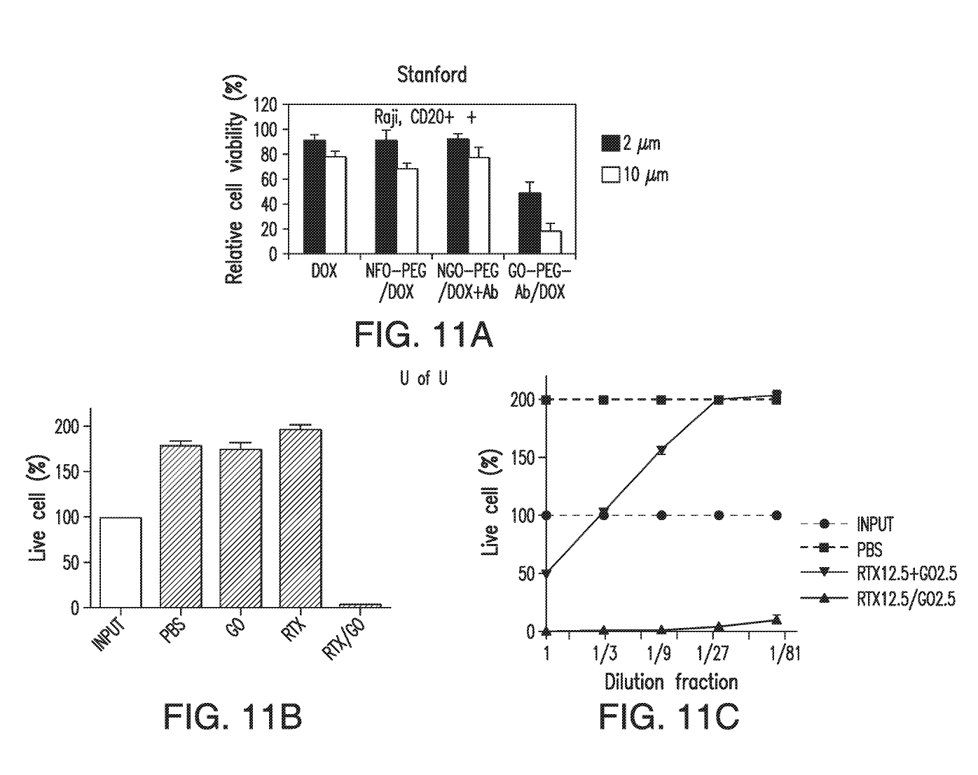

First, RTX/GO (non-covalent conjugation) is more potent in vitro compared to GO-PEG-RTX/DOX (covalent conjugation). For comparison, Raji cells (a B-cell lymphoma line) were cultured overnight with 10% PBS, GO (2 μg/ml), free RTX (10 μg/ml) or RTX/GO (10 μg/2 μg/ml) and analyzed by flow cytometry. Live versus dead cells were identified by the LIVE/DEAD cell dyes and the percentages of live cells relative to input cells of each treatment are shown in FIG. 11B). Raji cells were cultured overnight with decreasing concentrations of RTX/GO (bottom line, triangles) or free RTX plus free GO (RTX+GO, diagonal line, upside down triangles) added to the culture at initiation of culture. The starting concentration for free RTX and free GO was 12.5 μg/ml and 2.5 μg/ml respectively (FIG. 11C). The results using the covalent conjugation orientation show that GO-PEG-Ab/DOX is limited to inhibiting lymphoma (Raji) cell growth, even at high concentration (10 μM) of the cytotoxic drug DOX (FIG. 11A; (Sun et al. (2008) Nano Res. 1(3): 203-212)), suggesting that using GO-PEG-Ab/DOX (covalent conjugation) may be limited to slowing lymphoma progression in the treatment of patients. In contrast, exposure to RTX/GO (non-covalent conjugation) resulted in almost completely killed Raji cells in culture, even at low RTX/GO concentrations (FIG. 11B-C), suggesting that RTX non-covalently conjugated to GO is better at killing lymphoma cells, and that the administration of RTX/GO has the potential to cure lymphomas.

Second, RTX/GO is effective at killing target lymphoma cells with rapid kinetics, thus, eliminating the majority of lymphoma cells in culture by 30 minutes. It is well known that chemotherapeutic drugs kill lymphoma cells with slow kinetics such that no significant cell death is detectable until day 3 of culture. Given that the treatment regimen, GO-PEG-Ab/DOX, relies on the chemotherapeutic agent, DOX, to kill lymphoma cells, the killing kinetics may also be slow. Relying on slow killing kinetics may significantly impair the in vivo therapeutic capacity of GO-PEG-Ab/DOX, as the half-life of GO-PEG-Ab/DOX in the serum is expected to be shorter compared to free RTX and free DOX. At this time, there is no data available with respect to the in vivo therapeutic efficacy of GO-PEG-Ab/DOX since the results reported in Sun et al. (2008) Nano Res. 1(3): 203-212 are limited to in vivo studies.

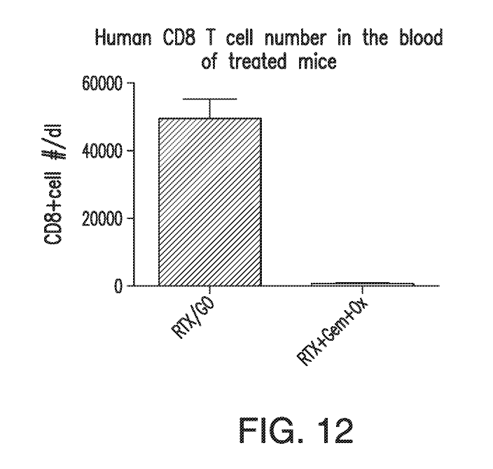

Third, the cytotoxicity exerted by RTX non-covalently bound to GO to cells is specific. RTX/GO kills CD20+ lymphoma cells, but does not affect T-cells involved in cellular immunity (FIG. 12). Lymphoma-bearing mice received human allogeneic lymphocytes and were treated with RTX/GO or RTX plus the chemotherapeutic drugs gemcitabine (Gem) and oxaliplatin (Ox) once every 2 days for 4 days. Human CD8 T cells in peripheral blood were enumerated one week after the last treatment. As treatments such as GO-PEG-Ab/DOX rely on chemotherapeutic drugs to attack lymphoma cells, these treatments, thus, may also attack immune system cells, thereby resulting in other non-specific effects. While chemotherapeutic drugs, such as DOX, may be concentrated at the lymphoma site(s) as a result of targeted delivery by GO-PEG-Ab/DOX (when in this case, the antibody used was RTX), the high concentration of such chemotherapeutic drugs may also make the lymphoma-infiltrating lymphocytes, composed mostly of tumor-specific T cells, particularly vulnerable to drug toxicity, thus, compromising antitumor immunity.

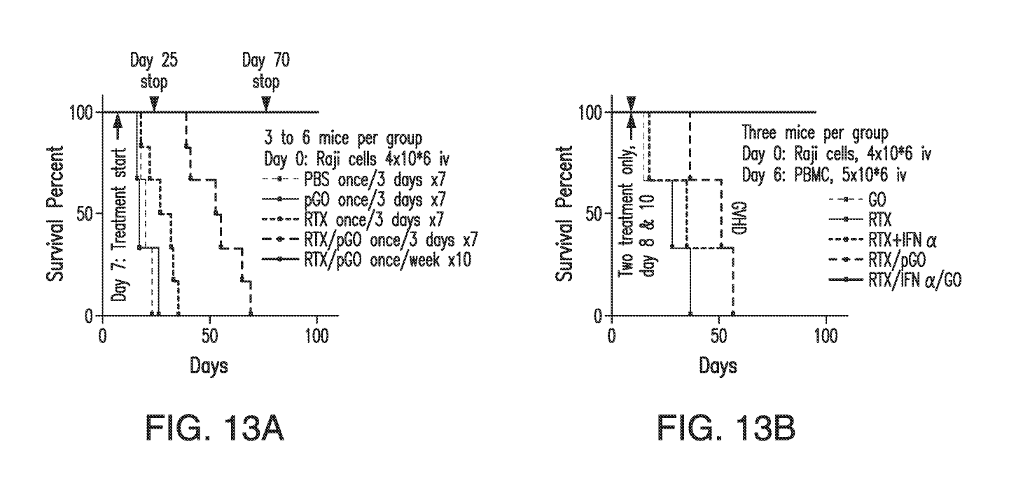

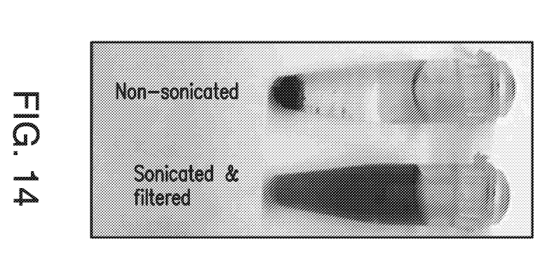

Fourth, RTX/GO has the capacity to eliminate lymphoma in vivo. The results described herein demonstrate that short periods (e.g., one to 3 weeks) of therapy in lymphoma-bearing mice administered intravenous RTX/GO result in rapid elimination of lymphoma and protection of the mice from death (FIGS. 5, 6 & 13A). The short period of therapy may be associated with late lymphoma relapse (FIG. 13A); however, extended periods (e.g., 10 weeks) of therapy results in indefinite remission, and may be curative (FIG. 13A). For these experiments, groups of mice were given the indicated treatment intravenously (iv) every 3 days for 21 days.

In the presence of transfused human lymphocytes, a minimum of two RTX/GO treatments prevented a relapse while lymphoma progressed in other treatment groups (FIG. 13B), demonstrating enhanced therapeutic efficacy of RTX/GO in the presence of lymphocytes. The human lymphocytes also caused xenogeneic graft-versus-host disease (GVHD) in the mice. Notably, associating IFNα with RTX/GO prevented GVHD, resulting in indefinite survival of the mice (FIG. 13B).

### Example 6: The Size of Graphene Oxide

The size of GO as made by a modified Hummers method varies. GO in its original form is unstable and precipitates rapidly in human serum (FIG. 14, left). To increase the stability of GO, GO was diluted to 1 mg/ml with 10% PBS and sonicated with a probe sonicator (Sonic Dismembrator, Fisher Scientific, Model 550) for 120 minutes at an amplitude of 4, followed by filtration through a 0.22 μm filter. Sonicated/filtered GO (sGO) remained in dispersion in human serum indefinitely (FIG. 14, right), indicating a substantial enhancement in stability.

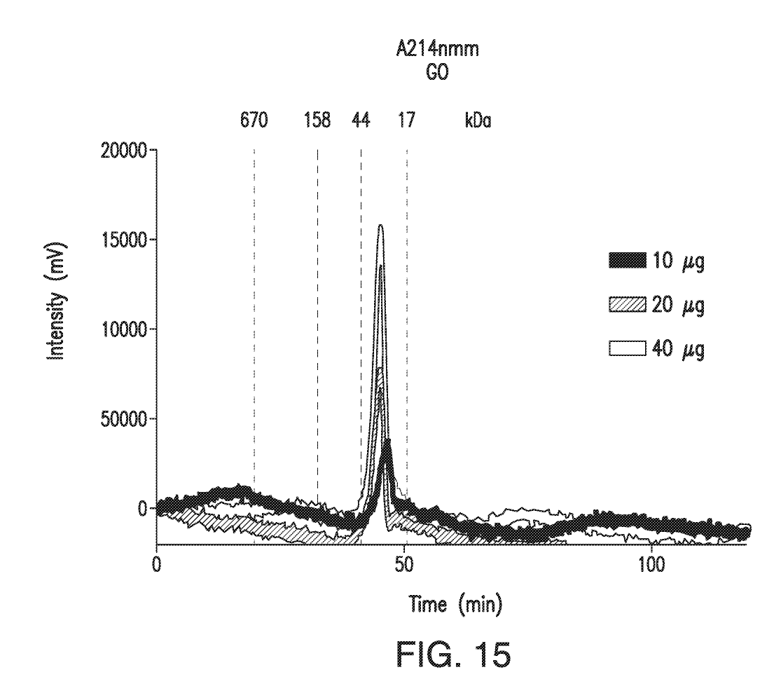

High performance liquid chromatography (HPLC) was used to measure molecular weight of sGO (FIG. 15). HPLC reveals that sGO is remarkably uniform in size with an estimated molecular weight of approximately 30 kDa, as compared to the reference molecules of known molecular weight (FIG. 15). Therefore, the molecular weight of sGO is about ⅕ of the molecular weight of trastuzumab (TRA, 148 kD).

### Example 7: Trastuzumab (TRA) can Stably be Associated with GO Through Non-Covalent Bonds

Consisting of sp2-hybridized carbon rings with hydroxyl and carboxyl groups. GO has the potential to non-covalently interact with antibody molecules through π-stacking, hydrophobic interactions, as well as with hydrogen and ionic bonds [8, 9].

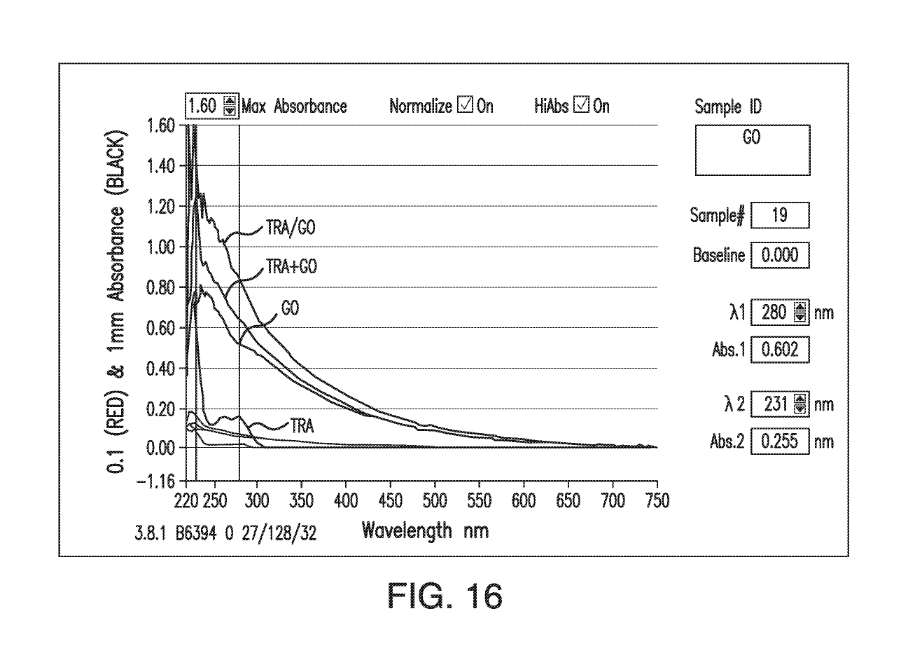

To determine whether TRA and GO can stably associate with each other through non-covalent bonds, vigorously sonicated and 0.221 μm-filtered GO (FIG. 16) was mixed with TRA in 10% PBS, and incubated at 37° C. overnight under constant agitation. On UV-Vis spectroscopy, free TRA absorption peaked at 280 nm, whereas free GO had a broad absorption spectrum peaking at 230 nm as previously reported [10]. The pre-incubated mixture of TRA and GO (referred to as TRA/GO) gave rise to an absorption spectrum similar to that of free GO but with substantially increased magnitude (FIG. 16), showing TRA is non-covalently conjugated to GO. When TRA and GO were mixed (TRA+GO) at the time of measurement, the magnitude of the absorbance was smaller compared to that of the pre-incubated mixture of TRA and GO (TRA/GO). This demonstrates that the stronger the light absorption, the stronger the association between TRA and GO.

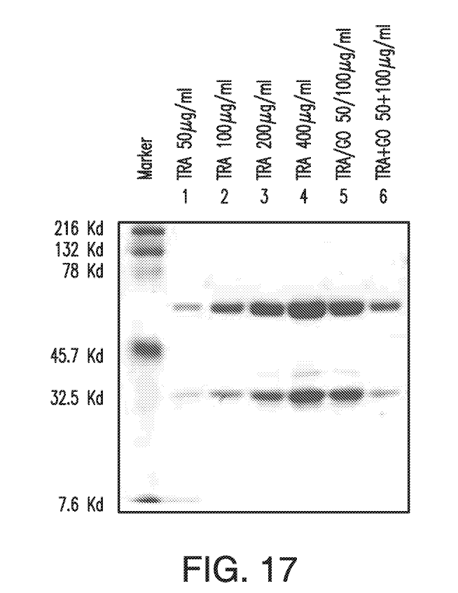

To quantitate the stoichiometric association between TRA and GO, TRA/GO mixture was made with TRA at 1000 μg/ml and GO at 50 μg/ml. After overnight incubation, the mixture was centrifuged to precipitate TRA-bound GO, which was then thoroughly washed with PBS in 37° C. to remove loosely bind TRA. The GO-bound TRA was eluted from GO with denaturing buffer and examined by SDS PAGE. As compared with the known concentrations of TRA loaded in parallel electrophoresis lanes, approximately 300 μg of TRA was found to be associated with 60 μg of GO (FIG. 17), giving rise to a 5:1 mass ratio of TRA to GO. When TRA and GO were incubated briefly (TRA+GO), a small amount of TRA was associated with GO. These results demonstrate that TRA and GO become stably associated after incubation under the above described conditions. TRA/GO does not dissociate when washed with buffered normal saline solutions at body temperature (37° C.), showing its potential stability when used in vivo.

### Example 8: GO Non-Covalently Conjugated to TRA Binds to HER2 with High Avidity

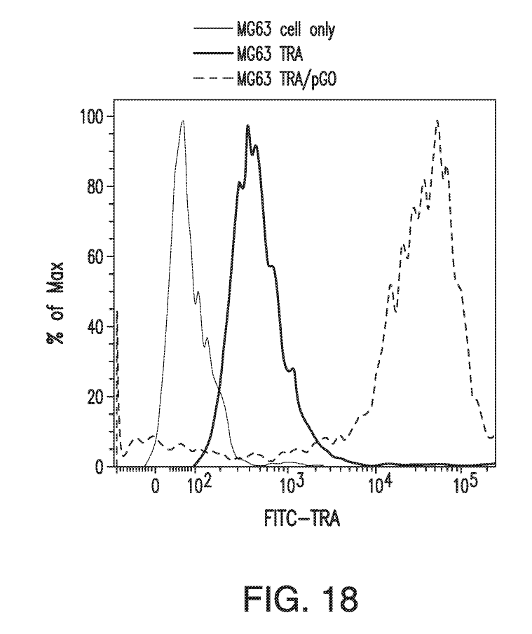

Since TRA and GO form a stable, non-covalent association through a stochastic process, such conjugation or association might interfere with the ability of TRA to bind HER2. To examine this possibility, FITC-conjugated to TRA was used to generate FITC-TRA/GO. FITC-TRA/GO was then used to stain a HER2-positive cell line, MG63, and FITC-TRA was used as a control. Both free FITC-TRA and FITC-TRA/GO positively stained MG63 cells, but the staining derived from FITC-TRA/GO was much (50-100 fold) brighter by flow cytometry (FIG. 18), indicating strong binding capacity of FITC-TRA/GO to HER2.

### Example 9: GO Non-Covalently Conjugated to TRA is Highly Cytotoxic to Osteosarcoma Cells

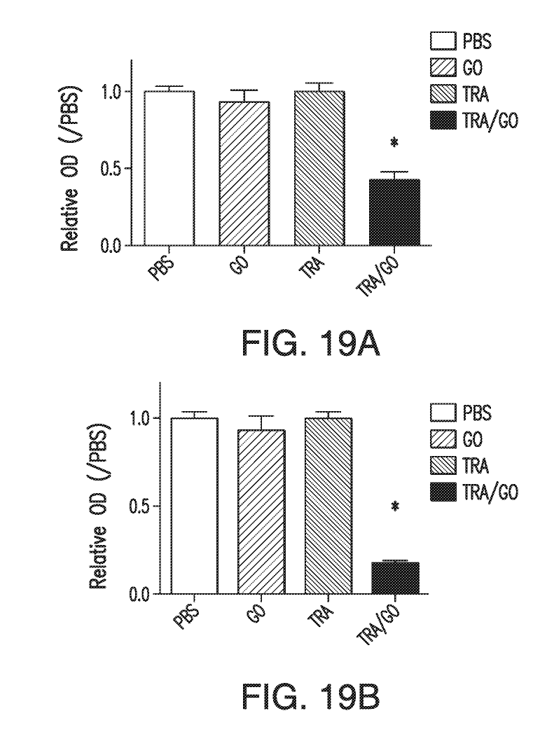

The results described herein show that the GO-associated anti-CD20 antibody rituximab (RTX/GO) kills CD20+ malignant lymphoma cells. The cytotoxicity of TRA/GO on HER2+ osteosarcoma cell lines, MG63 (FIG. 19A) and HOS (FIG. 19B) cells was then examined. MG63 and HOS cells were cultured overnight with TRA/GO, 10% PBS, GO or TRA and the percentage of live cells compared using the Cell Counting Kit (CCK8; Sigma-Aldrich). The cultures with TRA/GO had fewest viable cells while TRA alone did not affect proliferation of either MG63 or HOS cells. Flow cytometry was also used to identify live and dead cells using the LIVE/DEAD cell dyes (ThermoFisher) and revealed that TRA/GO killed about 50% of the target cells within four hours of culture (data not shown). These results demonstrate that TRA/GO has the capacity to kill HER2+ osteosarcoma cells while TRA alone is incapable.

Given the results of cell cultures showing that TRA/GO made with TRA to GO at a mass ratio of 5:1 gives rise to the highest cytotoxicity to HER2+ tumor cells, it can be determined that the optimal molar ratio of TRA to GO is 1:1, i.e., one TRA molecule to one GO molecule.

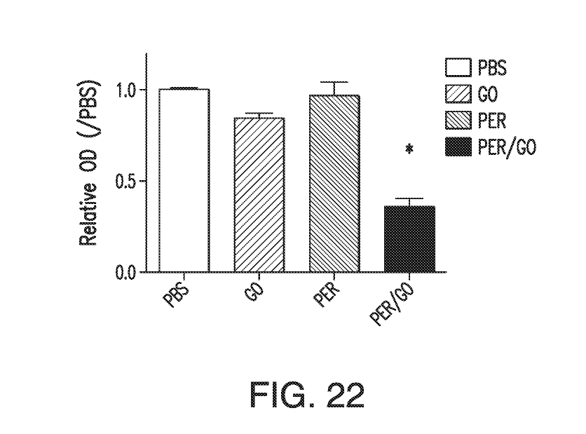

In a separate set of experiments, another anti-HER2 antibody, pertuzumab (PER), was non-covalently conjugated to GO. MG63 cells were cultured overnight with 10% PBS, GO, free PER or PER/GO and the percentage of live cells determined using the Cell Counting Kit-8 (CCK-8). The results show that pertuzumab non-covalently conjugated to GO (PER/GO) also potently kills MG63 cells (FIG. 22).

### Example 10: TRA/GO Causes Non-Apoptotic Cell Death

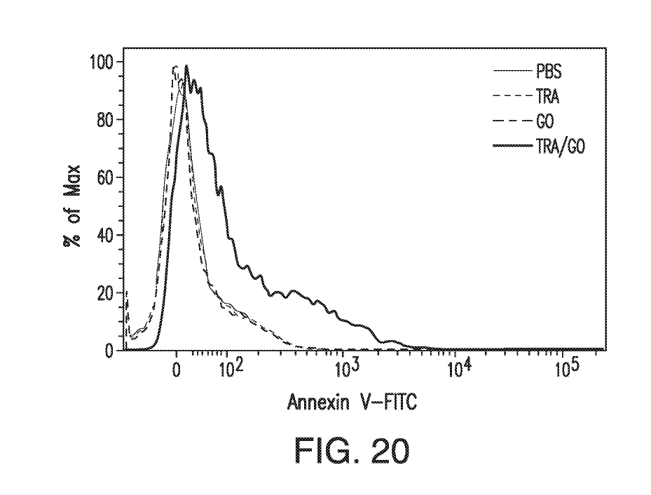

Annexin V did not significantly stain TRA/GO killed cells, suggesting that TRA/GO mediated cytotoxicity is through a non-apoptotic mechanism (FIG. 20). MG63 cells were cultured for 4 hours with PBS, TRA, GO or TRA/GO, stained with the apoptotic marker Annexin V and analyzed by flow cytometry. No gate was applied on displayed cells.

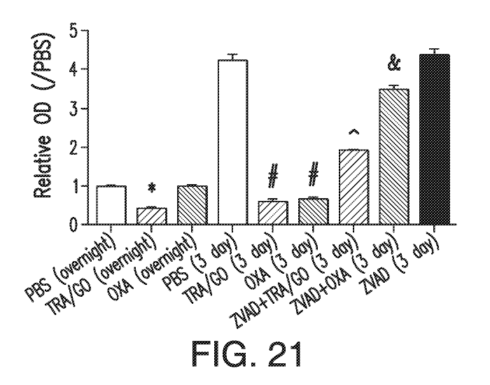

Consistent with this result, the pan-caspase inhibitor Z-VAD, which inhibits all the caspase activity of the apoptotic pathway, slightly alleviates TRA/GO-mediated cytotoxicity (FIG. 21). In addition, TRA/GO does not cause significant oxidative stress to the cells. Electronic microscopy and LDH release assays suggest that TRA/GO kills the osteosarcoma cells through damaging the plasma membrane. Oxa=oxaliplatin, *, #, ̂, and & indicate that the differences between the treatments and corresponding controls are statistically significant at p<0.05.

### Example 11: TRA/GO but not Free TRA Rapidly Eliminates Osteosarcoma In Vivo in a Xenograft Mouse Model

To study the therapeutic potential of TRA/GO in vivo, local and metastatic osteosarcoma was established by subcutaneous and intravenous transplantation of MG63 cells into immunodeficient NODrag^{ko}γ^{ko} (NRG) mice. NRG mice are deficient in T, B and NK cells with a defective complement system and impaired macrophage activity, and therefore constitute an animal model for the evaluation of therapeutic capacity of TRA/GO in the absence of host effector mechanisms, such as complement-dependent cytotoxicity or antibody-dependent cellular cytotoxicity.

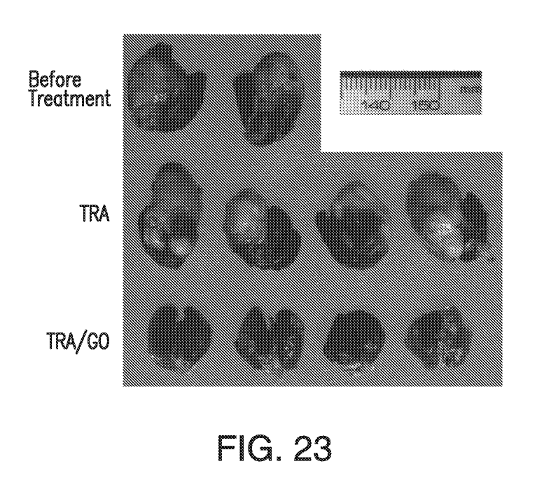

Subcutaneous inoculation of MG63 cells induces local tumors while intravenous inoculation gives rise to metastatic tumors in the lung, the most commonly involved organ by metastatic osteosarcoma in patients. Two weeks post inoculation, subcutaneous tumors became palpable, and metastatic tumors in the lungs were confirmed by pathological examination (FIG. 23). The mice then started receiving treatment with either TRA or TRA/GO twice a week. Two weeks after treatment, the mice receiving TRA become severely morbid and were sacrificed for analysis, along with TRA/GO-treated mice.

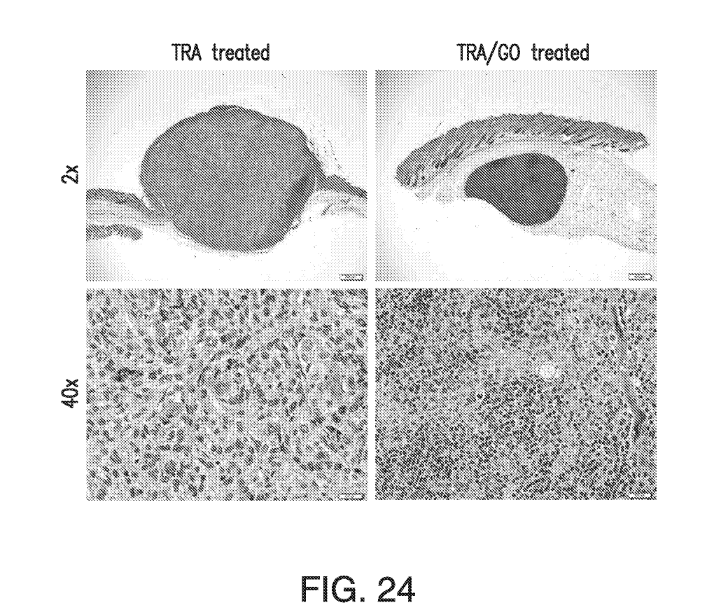

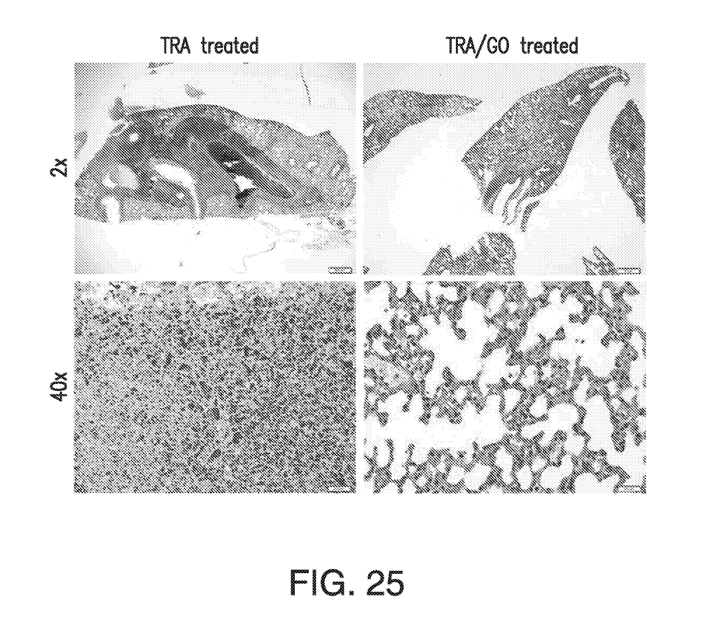

As shown in FIG. 24, large subcutaneous tumors were identified in the TRA-treated mice consisting of malignant spindle cells (FIG. 24, left). Although subcutaneous lesions were also identified in the TRA/GO-treated mice, those were smaller and composed of primarily inflammatory cells infiltrating the remaining degenerative tumor cells (FIG. 24, right). The lungs of all the TRA-treated mice contained massive tumors (FIG. 25). Under microscopic examination, these metastatic tumors in the lung were composed of highly malignant cells with malignant osteoid undergoing various degree of calcification; extensive necrosis was also present (FIG. 25, left). These histopathological features are typical of osteosarcoma seen in patients. In sharp contrast, there was no evidence of malignancy in the lungs of any of the TRA/GO-treated mice by thorough pathological examination, indicating that the tumors at this location were completely eliminated.

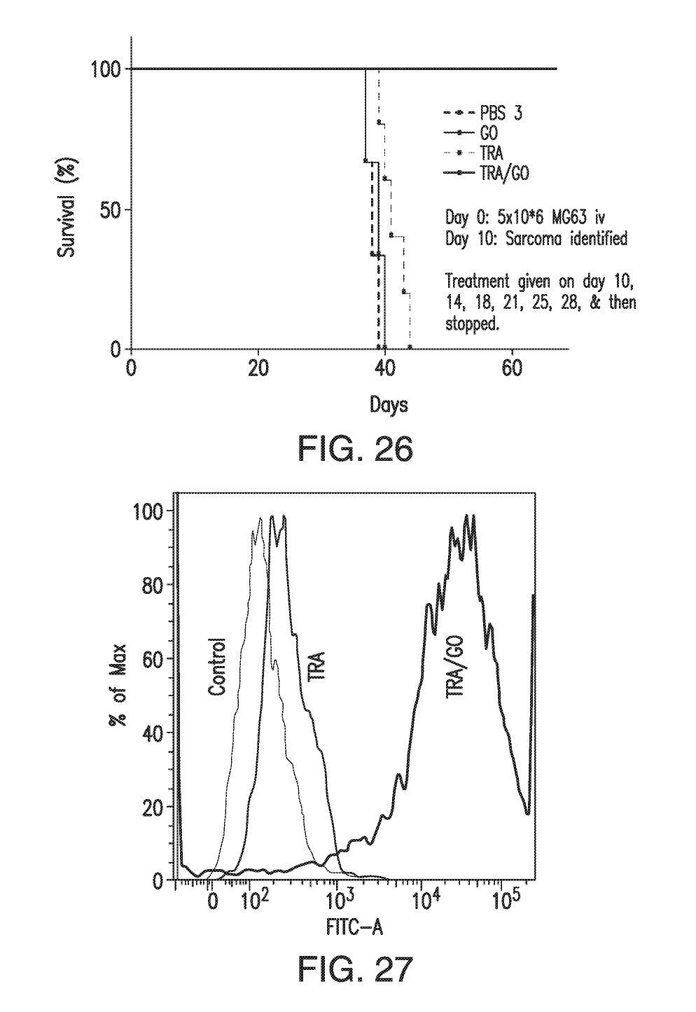

In survival experiments, mice, intravenously transplanted with MG63 cells, were given 6 treatments, with either PBS, GO, TRA or TRA/GO, as indicated in FIG. 26, starting 10 days after MG63 cell inoculation. All the mice that received PBS, GO or TRA died 10 to 15 days after the last treatment. Pathological examination revealed massive osteosarcoma in the lungs of all three groups of mice. All mice that received TRA/GO, however, remained completely healthy when sacrificed for analysis 40 days after the last treatment. Pathological examination showed no evidence of malignancy or pathological abnormalities in the lungs or other major organs that were thoroughly examined, further confirming the capacity of TRA/GO to eradicate MG63-derived osteosarcoma while TRA alone had little if any impact on propagation of the tumor in vivo.

Taken together, these data on MG63-derived osteosarcoma demonstrate that TRA/GO has the capacity to eliminate established osteosarcoma in the absence of chemotherapy or host immune effector mechanisms. Given the current lack of effective treatment for this dreadful tumor, these findings can have important clinical therapeutic implications, when non-surgical interventions including chemotherapy or irradiation are ineffective in controlling progression of metastatic osteosarcoma.

### Example 12: Therapeutic Potential of TRA/GO for Pancreatic Carcinoma

TRA/GO was tested for its ability to be cytotoxic on a well-characterized HER2+ pancreatic adenocarcinoma cell line, BxPC3 [26]. The results show that staining BxPC3 cells with FITC-TRA/GO gave rise to much brighter immunofluorescence as compared to free FTIC-TRA (FIG. 27), confirming the enhanced capacity of TRA/GO to bind to the cells.

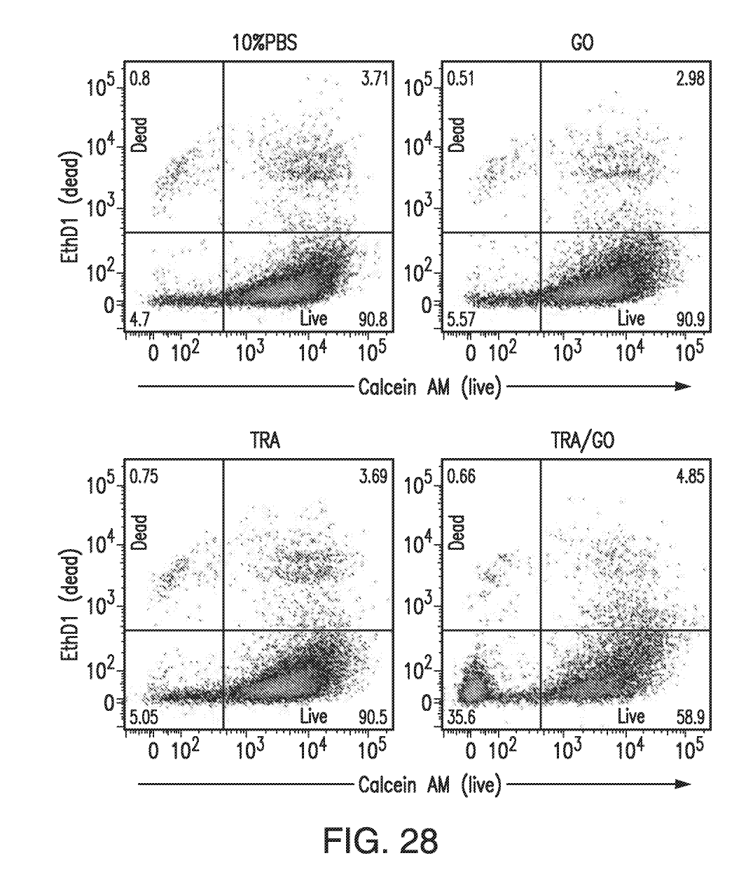

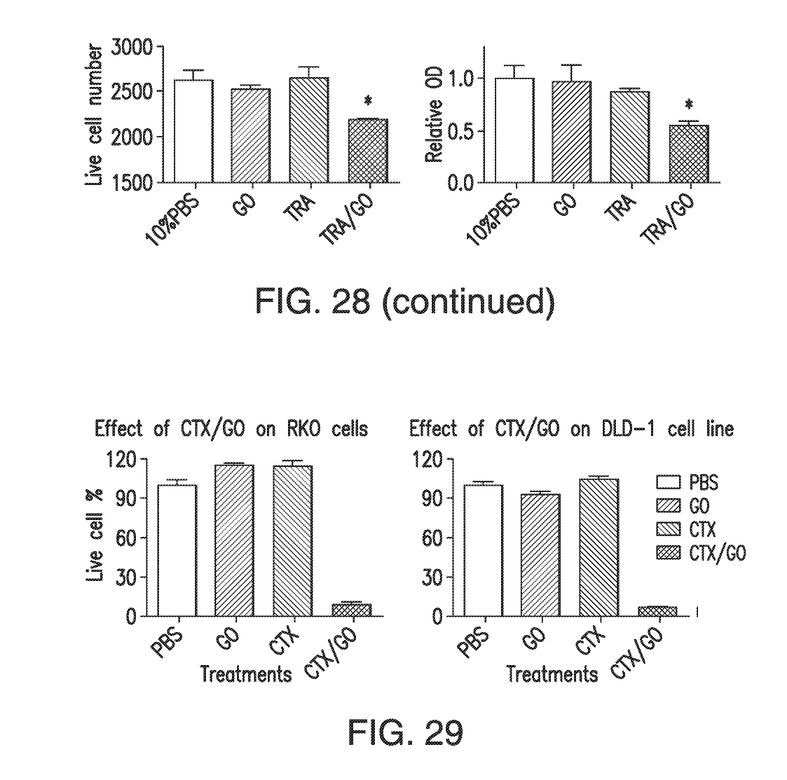

BxPC3 cells were stained with FITC-TRA at 50 μg/ml, or TRA/GO (50 μg/10 μg/ml). The cells were analyzed by flow cytometry. Treating BxPC3 cells in culture for 4 hours with TRA/GO killed BxPC3 cells while free TRA had no impact on viability of the cells (FIG. 28).

These in vitro results suggest that TRA/GO can be therapeutically effective against HER2+ pancreatic ductal carcinoma.

Taken together, the data presented herein on two types of malignancy, sarcoma and carcinoma, demonstrate that targeting HER2 using antibodies non-covalently conjugated to GO can effectively eliminate cancers cells while the original antibodies (e.g., antibodies not conjugated to GO) had no effect. These results suggest that targeting other HER2+ cancers, such as breast carcinoma, glioblastoma multiforme, etc, using anti-HER2 antibodies non-covalently conjugated to GO can be similarly effective as osteosarcoma or pancreatic ductal carcinoma.

### Example 13: Therapeutic Potential of Anti-EGFR1 Antibodies Non-Covalently Conjugated to GO for Treatment of Carcinomas of Colon or Lung

To improve anti-tumor capacity of anti-EGFR1 antibodies, cetuximab (CTX) was non-covalently conjugated to GO and the cytotoxicity of GO-associated CTX (CTX/GO) was studied on two colon carcinoma cell lines, RKO and DLD1. RKO carries wild-type KRAS, but DLD1 harbors mutated KRAS and is known to be resistant to CTX therapy. When the cells were cultured with free CTX or CTX/GO, free CTX had no cytotoxic effect on either cell line, but CTX/GO killed both RKO and DLD1 cells (FIG. 29). Colorectal carcinoma cell lines RKO and DLD1 were cultured overnight with 10% PBS. GO, free CTX or CTX/GO, and percentages of live cells were determined using the Cell Counting Kit-8 (CCK-8).

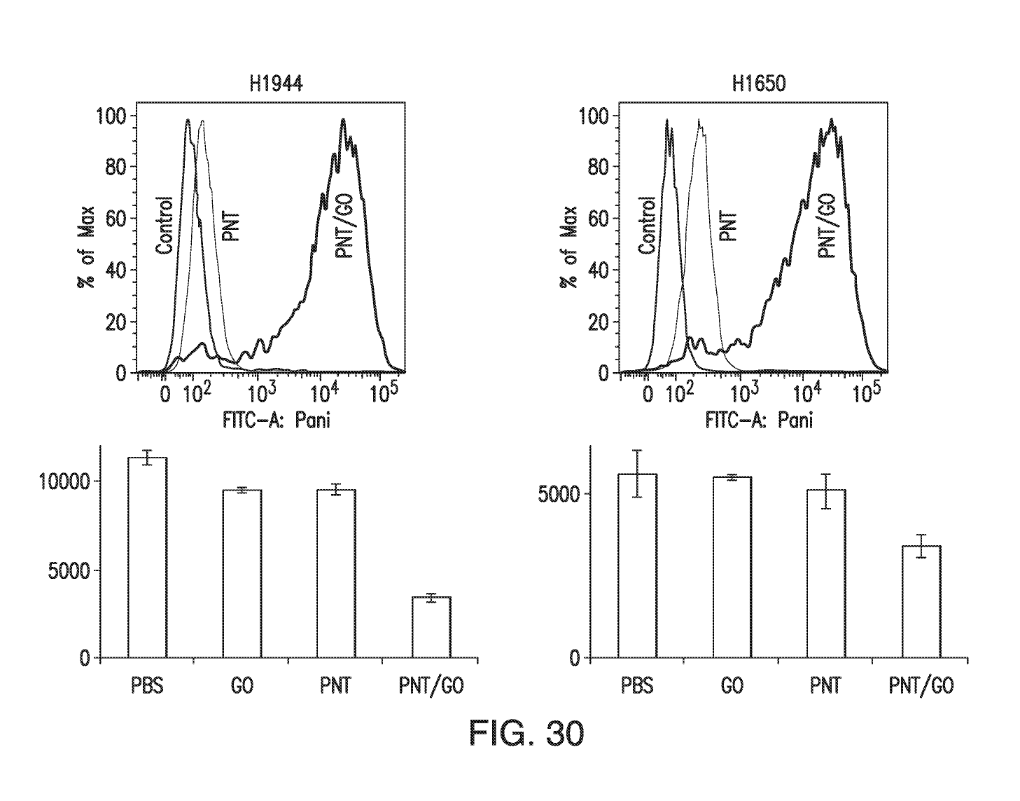

Another anti-EGFR1 antibody, panitumumab (PNT), was tested in original form or non-covalently conjugated to GO on two lung carcinoma cell lines, H1944 and H1650. H1944 expresses wild-type EGFR1 while H1650 has the in-frame deletion delE746-A750 mutation, which drives tumorigenesis. As shown in FIG. 30, PNT non-covalently conjugated to GO (PNT/GO) bound the cells with a stronger capacity compared to free PNT (FIG. 30, upper panel), and PNT/GO killed both types of carcinoma cells (FIG. 30, lower panel). H1944 and H1650 cells were stained with FITC-PNT or FITC-PNT/GO and analyzed by flow cytometry (upper panel). PNT/GO on lung carcinoma cell lines, H1944 and H1650, were cultured overnight with 10% PBS, GO, free PNT or PNT/GO, and percentages of live cells were determined using the Cell Counting Kit-8 (CCK-8) (FIG. 30, lower panel).

As EGFR1 is overexpressed on a large variety of epithelial malignancies (e.g., carcinomas), these results show that anti-EGFR1 antibodies non-covalently conjugated to GO have significant potential to effectively treat carcinomas from many different organs/locations in addition to the colon/rectum or lungs. Non-covalent conjugation of these antibodies to GO makes the antibodies directly cytotoxic, and also enables the antibody to kill KRAS-mutated tumors as well as EGFR+ carcinoma cells regardless of mutation or activation status of EGFR1.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

### Example 14: Trastuzumab Non-Covalently Conjugated to GO is More Potent than FDA-Approved Kadcyla®

In an effort to enhance the cytotoxicity of trastuzumab (TRA; also referred to as Herceptin®), TRA was linked to the cytotoxic agent emtansin to produce ado-trastuzumab emtansin (Kadcyla®). Kadcyla® is an FDA-approved for the treatment of breast carcinoma patients that are not responsive to TRA plus chemotherapy. Two osteosarcoma cell lines, MG63 and HOS, were used to compare the cytotoxic effects of Kadcyla® and TRA-GO. For this experiment, the cells were cultured with 10% PBS. TRA alone. Kadcyla® or TRA/GO for one day and then stained with live cell dye and examined using fluorescence microscopy. The results show that Kadcyla® exhibited limited cytotoxicity whereas TRA-GO displayed marked cytotoxicity in vitro.

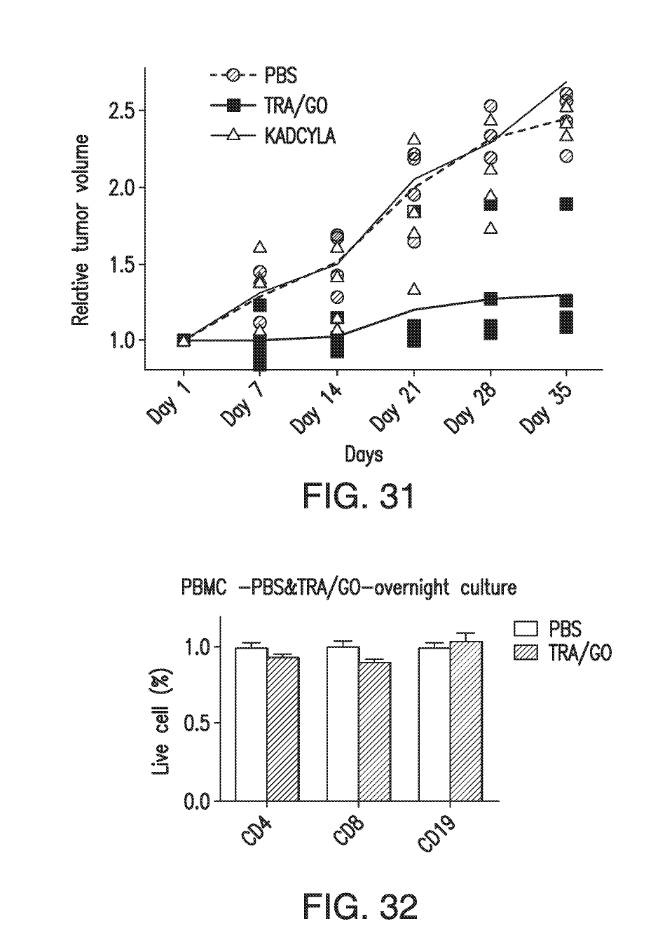

Next, immunodeficient NSG mice bearing subcutaneous osteosarcoma (derived from osteosarcoma cell line MG63) were intravenously administered PBS (control), TRA/GO or Kadcyla® twice a week. The results demonstrate that TRA-GO inhibits tumor growth, while Kadcyla® showed limited therapeutic effect (FIG. 31).

### Example 15: Trastuzumab Non-Covalently Conjugated to GO is More Potent than Trastuzumab or GO Alone

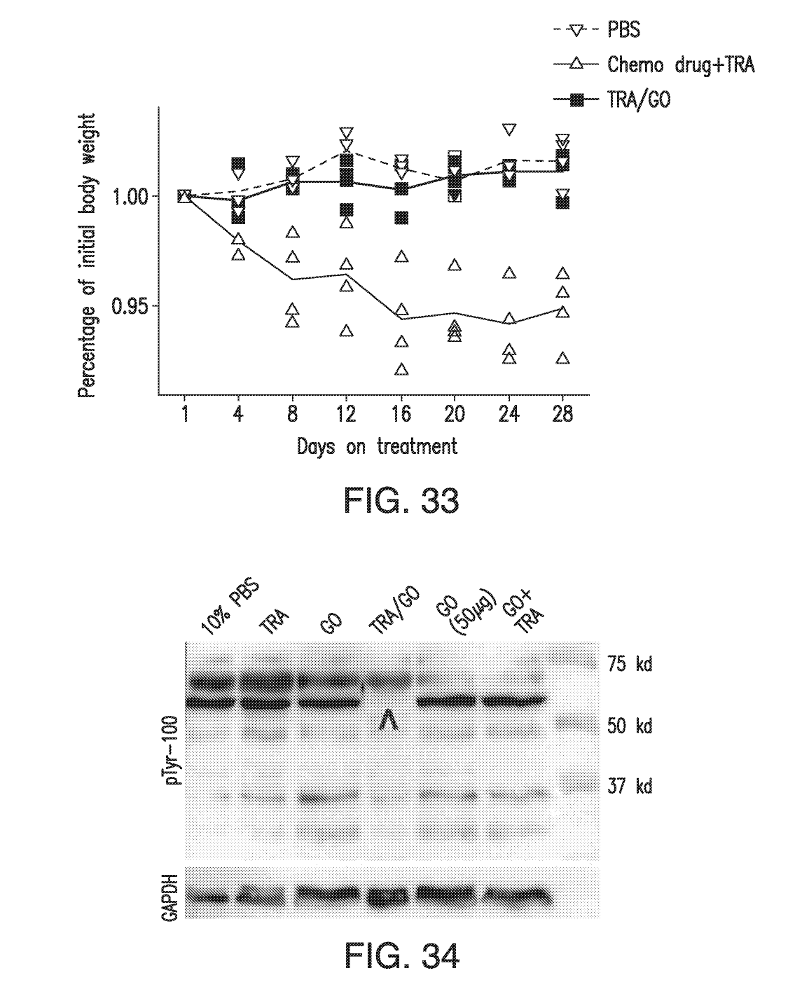

Additional analysis showed that TRA/GO did not affect human lymphocyte viability (FIG. 32) or cause any appreciable side effects in mice (FIG. 33). For instance, the number of CD4, CD8 and CD19 positive lymphocytes remained unchanged after overnight culture with PBS or TRA/GO. Further, mice intravenously administered a chemo drug plus TRA twice a week for twenty-eight days led to marked weight loss while mice treated with TRA/GO maintained their body weight similar to those mice treated with PBS as a control.

Intravenous administration of TRA/GO into immunodeficient NSG mice harboring MG63-derived osteosarcoma quickly eradicated the established osteosarcoma within 2 weeks, leading to indefinite survival of the animals, while the mice treated with TRA alone or PBS as a control succumbed to tumor progression. For example, large subcutaneous and lung tumors were identified in PBS-, GO- or TRA-treated mice but not TRA/GO-treated mice. The minute subcutaneous nodules and pale areas observed in the lungs of TRA/GO-treated mice were not actual tumors but rather chronic inflammation as revealed by microscopy. Upon microscopic analysis, the tumors identified in the PBS-, GO- and TRA-treated mice were osteosarcoma while the small subcutaneous nodules found in the TRA/GO-treated mice were granulomas; and the patchy gray areas in the lungs were chronic inflammation and atelectasis. Next, a Kaplan-Meier curve was generated. For this, 5×10⁶ MG63 cells were transplanted intravenously on day 0. Osteosarcoma was confirmed on day 14, the same day treatment started. Treatment with PBS, GO, TRA or TRA/GO continued every three days for a total of six treatments (day 32). All of the mice that received PBS, GO or TRA died of osteosarcoma while the TRA/GO-treated mice lived until they were sacrificed and in were in good condition to that point. When the mice were sacrificed on experimental day 70, no tumor was identified in any of the mice.

### Example 16: Trastuzumab Non-Covalently Conjugated to GO is Necrotic to Cells

As shown herein, RTX/GO kills target cells by necrosis rather than by apoptosis. TRA/GO also is also cytotoxic to target cells via necrosis. To further analyze the cells for specific features characteristic of either necrosis or apoptosis, the Burkitt lymphoma cell line Daudi was treated in culture for 8 hours with RTX/GO or PBS as control. Using electronic microscopy, TRG/GO was visualized as approximately 40×200 nm nano-flakes, some of which were bound to cells. RTX/GO-bound cells showed ruptured cytoplasmic membrane with edematous cytoplasm and loss of integrity of the organelles and the nucleus appeared intact, a finding that is consistent with necrosis cell death, not apoptosis. These results were confirmed in an electronic microscopy analysis using the osteosarcoma cell line MG63. Similar to the results described herein, the PBS-treated cells and the TRA/GO-treated cells were bound by TRA/GO nanoflakes, and the dead cells showed cytoplasmic swelling, an increased number of autophagic vacuoles, organelle degeneration, and plasma membrane rupture, features of necrosis or necrotic cell death.

Fluorescent microscopy and flow cytometry analysis confirm that TRA/GO-mediated cytotoxicity in MG63 cells is not due to apoptosis. The results show, for example, that a pan-caspase inhibitor, z-VAD, does not affect TRA/GO-mediated cytotoxicity, suggesting non-apoptotic cell death. In contrast, necrostatin-1 (Nec1), a specific inhibitor of RIPK1 or an MLKL inhibitor, mitigated the cell death, indicating that TRA/GO kills target cells by necroptosis (e.g., a programmed form of necrosis, or inflammatory cell death). TRA/GO, but not GO or TRA alone, induces oxidative stress (i.e., production of reactive oxygen species, ROS) in target cells. Treatment with a reducing agent, Tiron, blocked ROS production and protected the cells from death, suggesting that ROS is required for the TRA/GO-mediated cytotoxic activity. Treating target cells with W6-32/GO, a monoclonal antibody specific for class I major histocompatibility complex also induced strong ROS production, but did not result in cell death, indicating that ROS production alone is insufficient to activate necroptosis, and that simultaneous HER2 signaling may be necessary for cytotoxicity.

Western blots generated from lysates of MG63 cells cultured for 5 minutes with PBS. TRA, GO, TRA/GO, GO (50 μg) and GO+TRA resulted in a complete loss of a major tyrosine-phosphorylated protein species (FIG. 34), suggesting that TRA/GO induced rapid, strong intracellular signaling upon binding to the HER2 receptor. In contrast, TRA, GO (including 50 μg/ml high concentration of GO that induces ROS, e.g., oxidative stress), or GO plus TRA (that were separately added to the culture) did not cause loss of this tyrosine-phosphorylated protein, indicating that association between TRA and GO is responsible for the cytotoxic effects to cells. Blocking HER2 signaling with lapatinib (also referred to as Tykerb®), a small molecule inhibitor of HER2 tyrosine kinase activity, abrogated the killing, confirming that it is the combination of TRA non-covalently conjugated to GO that is important for the cytotoxici effects described herein.

These results demonstrate that TRA/GO kills osteosarcoma cells through necroptosis. The execution of necroptosis relies on the capacity of TRA/GO to simultaneously induce oxidative stress and rapid, detrimental signaling through HER2 in the target cells. This uncommon property makes TRA/GO an extraordinary, effective killer of HER2+ osteosarcoma cells. Thus, similar to the results with RTX/GO (29), TRA/GO demonstrates much stronger anti-cancer activity as compared to free TRA.

### Example 17: RTX Non-Covalently Conjugated to GO is Cytotoxic to Z-138 Cells

Mantel cell lymphoma is a distinct lymphoma type for which no effective treatment is currently available. RTX/GO cytotoxicity is examined using a marginal lymphoma cell line Z-138. Mantel cell lymphoma cell line Z-138 was cultured with 10% PBS, RTX, GO or RTX/GO for 48 hours and stained with live cell dye that marks dead cells. Similar to other Examples described herein, and using fluorescence microscopy to examine the Z-138 cell cultures, the results show that RTX/GO killed Z-138 cells while RTX alone and GO alone showed a similar result to cells treated with PBS (control).

### REFERENCES

1. Onda M, Matsuda S, Higaki S, Iijima T, Fukushima J. Yokokura A, Kojima T, Horiuchi H, Kurokawa T and Yamamoto T. ErbB-2 expression is correlated with poor prognosis for patients with osteosarcoma. Cancer. 1996; 77(1):71-78.
2. Gorlick R. Huvos A G, Heller G. Aledo A, Beardsley G P, Healey J H and Meyers P A. Expression of HER2/erbB-2 correlates with survival in osteosarcoma. J Clin Oncol. 1999; 17(9):2781-2788.
3. Akatsuka T, Wada T, Kokai Y. Kawaguchi S, Isu K. Yamashiro K, Yamashita T, Sawada N. Yamawaki S and Ishii S. ErbB2 expression is correlated with increased survival of patients with ostcosarcoma. Cancer. 2002; 94(5): 1397-1404.
4. Akatsuka T. Wada T, Kokai Y, Sawada N, Yamawaki S and Ishii S. Loss of ErbB2 expression in pulmonary metastatic lesions in osteosarcoma. Oncology. 2001; 60(4):361-366.
5. Anninga J K, van de Vijver M J, Cleton-Jansen A M, Kristel P M, Taminiau A H, Nooij M, Egeler R M and Hogendoom P C. Overexpression of the HER-2 oncogene does not play a role in high-grade osteosarcomas. Eur J Cancer. 2004; 40(7):963-970.
6. Scotlandi K. Manara M C, Hattinger C M, Benini S, Perdichizzi S, Pasello M, Bacci G, Zanella L, Bertoni F, Picci P and Serra M. Prognostic and therapeutic relevance of HER2 expression in osteosarcoma and Ewing's sarcoma. Eur J Cancer. 2005; 41(9): 1349-1361.
7. Ebb D, Meyers P, Grier H, Bemstein M, Gorlick R. Lipshultz S E, Krailo M, Devidas M, Barkauskas D A, Siegal G P. Ferguson W S, Letson G D, Marcus K. Goorin A, Beardsley P and Marina N. Phase 11 trial of trastuzumab in combination with cytotoxic chemotherapy for treatment of metastatic osteosarcoma with human epidermal growth factor receptor 2 overexpression: a report from the children's oncology group. J Clin Oncol. 2012; 30(20):2545-2551.
8. Geim A K and Novoselov K S. The rise of graphene. Nature materials. 2007; 6(3):183-191.
9. Yang K, Li Y, Tan X, Peng R and Liu Z. Behavior and toxicity of graphene and its functionalized derivatives in biological systems. Small. 2013; 9(9-10): 1492-1503.
10. Huang P, Xu C, Lin J, Wang C, Wang X. Zhang C, Zhou X, Guo S and Cui D. Folic Acid-conjugated Graphene Oxide loaded with Photosensitizers for Targeting Photodynamic Therapy. Theranostics. 2011; 1:240-250.
11. Bourguignon L Y and Singer S J. Transmembrane interactions and the mechanism of capping of surface receptors by their specific ligands. Proc Natl Acad Sci USA. 1977; 74(11):5031-5035.
12. Taylor R B, Duffus W P, Raff M C and de Petris S. Redistribution and pinocytosis of lymphocyte surface immunoglobulin molecules induced by anti-immunoglobulin antibody. Nat New Biol. 1971; 233(42):225-229.
13. Baxter A G and Cooke A. Complement lytic activity has no role in the pathogenesis of autoimmune diabetes in NOD mice. Diabetes. 1993; 42(11): 1574-1578.
14. Shultz L D, Schweitzer P A, Christianson S W, Gott B, Schweitzer I B, Tennent B. McKenna S. Mobraaten L, Rajan T V, Greiner D L and et al. Multiple defects in innate and adaptive immunologic function in NOD/LtSz-scid mice. Journal of immunology. 1995; 154(1):180-191.
15. Jemal A, Siegel R, Xu J and Ward E. Cancer statistics, 2010. C A Cancer J Clin. 2010; 60(5):277-300.
16. Conroy T, Desseigne F, Ychou M, Bouche O, Guimbaud R, Becouarn Y, Adenis A, Raoul J L, Gourgou-Bourgade S, de la Fouchardiere C, Bennouna J, Bachet J B, Khemissa-Akouz F, Pere-Verge D, Delbaldo C, Assenat E, et al. FOLFIRINOX versus gemcitabine for metastatic pancreatic cancer. N Engl J Med. 2011; 364(19):1817-1825.
17. Philip P A. Benedetti J, Corless C L, Wong R, O'Reilly E M, Flynn P J, Rowland K M, Atkins J N, Mirtsching B C, Rivkin S E, Khorana A A, Goldman B, Fenoglio-Prciser C M, Abbruzzese J L and Blanke C D. Phase III study comparing gemcitabine plus cetuximab versus gemcitabine in patients with advanced pancreatic adenocarcinoma: Southwest Oncology Group-directed intergroup trial S0205. J Clin Oncol. 2010; 28(22):3605-3610.
18. Saxby A J, Nielsen A. Scarlett C J, Clarkson A, Morey A, Gill A and Smith R C. Assessment of HER-2 status in pancreatic adenocarcinoma: correlation of immunohistochemistry, quantitative real-time RT-PCR, and FISH with aneuploidy and survival. Am J Surg Pathol. 2005; 29(9): 1125-1134.
19. Tobita K. Kijima H, Dowaki S, Kashiwagi H, Ohtani Y, Oida Y, Yamazaki H, Nakamura M, Ueyama Y, Tanaka M. Inokuchi S and Makuuchi H. Epidermal growth factor receptor expression in human pancreatic cancer: Significance for liver metastasis. Int J Mol Med. 2003; 11(3):305-309.
20. Dugan M C, Dergham S T, Kucway R, Singh K, Biemat L. Du W, Vaitkevicius V K, Crissman J D and Sarkar F H. HER-2/neu expression in pancreatic adenocarcinoma: relation to tumor differentiation and survival. Pancreas. 1997; 14(3):229-236.
21. Chou A, Waddell N, Cowley M J, Gill A J, Chang D K, Patch A M, Nones K, Wu J, Pinese M, Johns A L. Miller D K, Kassahn K S, Nagrial A M, Wasan H, Goldstein D. Toon C W, et al. Clinical and molecular characterization of HER2 amplified-pancreatic cancer. Genome Med. 2013; 5(8):78.
22. Yamanaka Y. Friess H. Kobrin M S, Buchler M, Kunz J, Beger H G and Kore M. Overexpression of HER2/neu oncogene in human pancreatic carcinoma. Hum Pathol. 1993; 24(10):1127-1134.
23. Safran H. Iannitti D, Ramanathan R, Schwartz J D, Steinhoff M, Nauman C, Hesketh P, Rathore R, Wolff R, Tantravahi U, Hughes T M, Maia C, Pasquariello T, Goldstein L, King T, Tsai J Y, et al. Herceptin and gemcitabine for metastatic pancreatic cancers that overexpress HER-2/neu. Cancer Invest. 2004; 22(5):706-712.
24. Walsh N, Kennedy S, Larkin A, Corkery B, O'Driscoll L, Clynes M, Crown J and O'Donovan N. EGFR and HER2 inhibition in pancreatic cancer. Invest New Drugs. 2013; 31(3):558-566.
25. Assenat E, Azria D. Mollevi C, Guimbaud R, Tubiana-Mathicu N, Smith D, Delord J P, Samalin E, Portales F, Larbouret C, Robert B. Bibeau F, Bleuse J P, Crapez E, Ychou M and Pelegrin A. Dual targeting of HER1/EGFR and HER2 with cetuximab and trastuzumab in patients with metastatic pancreatic cancer after gemcitabine failure: results of the “THERAPY” phase 1-2 trial. Oncotarget. 2015; 6(14):12796-12808.
26. Larbouret C. Gaborit N. Chardes T, Coelho M, Campigna E, Bascoul-Mollevi C, Mach J P, Azria D, Robert B and Pelegrin A. In pancreatic carcinoma, dual EGFR/HER2 targeting with cetuximab/trastuzumab is more effective than treatment with trastuzumab/erlotinib or lapatinib alone: implication of receptors' down-regulation and dimers' disruption. Neoplasia. 2012; 14(2): 121-130.
27. Amado R G, Wolf M, Peeters M. Van Cutsem E. Siena S, Freeman D J, Juan T. Sikorski R, Suggs S, Radinsky R, Patterson S D and Chang D D. Wild-type KRAS is required for panitumumab efficacy in patients with metastatic colorectal cancer. J Clin Oncol. 2008; 26(10): 1626-1634.
28. Karapetis C S, Khambata-Ford S, Jonker D J, O'Callaghan C J, Tu D, Tebbutt N C, Simes R J, Chalchal H, Shapiro J D, Robitaille S. Price T J, Shepherd L, Au H J, Langer C, Moore M J and Zalcberg J R. K-ras mutations and benefit from cetuximab in advanced colorectal cancer. N Engl J Med. 2008; 359(17):1757-1765.
29. Luo, C., Z. Deng, L. Li, F. Clayton, A. L. Chen, R. Wei, R. Miles, D. M. Stephens, M. Glenn, X. Wang, P. E. Jensen, and X. Chen. 2016. Association of rituximab with graphene oxide confers direct cytotoxicity for CD20-positive lymphoma cells. Oncotarget 7: 12806-12822.

What is claimed is:

1. A composition comprising one or more antibodies non-covalently conjugated to graphene oxide.

2. The composition of claim 1, wherein the antibody is an anti-CD20 monoclonal antibody.

3. The composition of claim 2, wherein the anti-CD20 monoclonal antibody is ofatumumab, rituximab, tositumomab, obinutuzumab, ibritumomab or a biologically active variant thereof.

4. The composition of claim 3, wherein the anti-CD20 monoclonal antibody is rituximab.

5. The composition of claim 1, wherein the graphene oxide is functionalized.

6. The composition of claim 1, wherein the composition is multivalent.

7. The composition of claim 1, wherein the one or more antibodies and graphene oxide are present in a mass ratio of 5:1.

8. The composition of claim 1, wherein the one or more antibodies comprises one or more monomers.

9. The composition of claim 8, wherein the one or more monomers are non-covalently bound to the graphene oxide thereby forming a polymer of antibodies.

10. The composition of claim 1, further comprising a hydrophilic polymer, wherein the polymer is polyethylene glycol.

11. The composition of claim 1, wherein the non-covalent conjugation is through pi-stacking, hydrophobic interaction, ionic binding or hydrogen binding.

12. The pharmaceutical composition comprising the composition of claim 1 and a pharmaceutically acceptable carrier.

13. The pharmaceutical composition comprising the composition of claim 1, wherein the pharmaceutical composition is formulated for intravenous administration, intratumor injection, or into peritoneal or pleural cavity.

14. A method of treating a cancer, the method comprising:
(a) identifying a patient in need of treatment;
(b) administering to the patient a therapeutically effective amount of the composition of claim 1; and
(c) a pharmaceutically acceptable carrier.

15. The method of claim 14, wherein the anti-CD20 monoclonal antibody is ofatumumab, rituximab, tositumomab, obinutuzumab, ibritumomab or a biologically active variant thereof.

16. The method of claim 15, wherein the anti-CD20 monoclonal antibody is rituximab.

17. The method of claim 14, wherein the patient is a human patient.

18. The method of claim 14, wherein the cancer is a primary, secondary, refractory, or relapsing tumor.

19. The method of claim 18, wherein the primary, secondary, refractory, or relapsing tumor is a blood cell tumor.

20. The method of claim 19, wherein the blood cell tumor is lymphoma.

21. The method of claim 20, wherein the lymphoma is a non-Hodgkin's lymphoma.

22. The method of claim 21, wherein the non-Hodgkin's lymphoma is follicular lymphoma, mantle cell lymphoma, marginal zone cell lymphoma, diffuse large-B-cell lymphoma or Burkitt lymphoma.

23. The method of claim 14, wherein the cancer is associated with expression of CD20.

24. The method of claim 14, further comprising administering to the patient a therapeutically effective amount of radiation therapy, immunotherapy or chemotherapy or a combination thereof.

25. A composition for delivery of one or more antibodies to a cell, comprising:
(a) graphene oxide; and
(b) one or more antibodies non-covalently bound to the graphene oxide, wherein the non-covalent binding induces a conformational change in graphene oxide.

26. The composition for delivery of claim 25, wherein the one or more antibodies is a plurality of monomers.

27. The composition for delivery of claim 25, further comprising a polymer, wherein the polymer is polyethylene glycol.

28. The composition for delivery of claim 25, wherein the antibody is an anti-CD20 monoclonal antibody.

29. The composition for delivery of claim 28, wherein the anti-CD20 monoclonal antibody is ofatumumab, rituximab, tositumomab, obinutuzumab, ibritumomab or a biologically active variant thereof.

30. The composition for delivery of claim 29, wherein the anti-CD20 monoclonal antibody bound to the graphene oxide is multivalent.

31. The composition for delivery of claim 25, wherein the antibody is bound to the graphene oxide through a non-covalent interaction including pi stacking, hydrophobic interaction, ionic bond or hydrogen bond.

32. A cell comprising the composition of claim 1.

33. A kit comprising a composition, wherein the composition comprises graphene oxide and one or more antibodies, wherein the graphene oxide is non-covalently bound to the one or more antibodies; and instructions for using the composition.

34. The kit of claim 33, wherein the composition is formed in the presence of a low salt solution.

35. The kit of claim 33, further comprising one or more items selected from the group consisting of a sterile fluid, a syringe and a sterile container.

36. The kit of claim 33, wherein the composition further comprises a pharmaceutically acceptable carrier.

37. A method of preparing a nanomaterial complex for delivery of a plurality of antibodies to a cell, the method comprising:
(a) preparing the nanomaterial through a functionalization process, wherein the nanomaterial is a graphene oxide sheet;
(b) attaching a plurality of antibody monomers to the graphene oxide sheet, wherein the loading is through non-covalent binding;
(c) incubating the antibody monomers with the graphene oxide sheet in a low salt solution; and
(d) forming a stable aqueous dispersion of the nanomaterial complex.

38. The method of claim 37, wherein the non-covalent binding is through pi-stacking.

39. The method of claim 37, wherein the low salt solution has a concentration of 10% PBS containing 0.09% NaCl.

40. The method of claim 37, wherein the antibodies to graphene oxide are present in a mass ratio of 5:1.

41. A method of delivering one or more antibodies to a cell, comprising the composition of claim 1, the method comprising contacting the cell with the composition for a sufficient time to permit crosslinking of the antibody to the cell.

42. The method of claim 41, wherein the antibodies have increased dissociation at low pH.

43. The method of claim 41, further comprising the step of contacting the composition with serum, wherein the composition does not dissociate in the serum.

44. A molecular probe comprising the composition of claim 1, further comprising a detectable label.

45. The molecular probe of claim 45, wherein the label is attached to the therapeutic agent.

48. The composition of claim 1, wherein the antibody is an anti-HER2 monoclonal antibody.

49. The composition of claim 48, wherein the anti-HER2 monoclonal antibody is trastuzumab and/or pertuzumab.

50. The composition of claim 1, wherein the antibody is an anti-HER1 monoclonal antibody.

51. The composition of claim 50, wherein the anti-HER1 monoclonal antibody is cetuximab and/or panitumumab.

52. A method of treating a cancer, the method comprising:
(a) identifying a patient in need of treatment;
(b) administering to the patient a therapeutically effective amount of the composition of claim 48; and
(c) a pharmaceutically acceptable carrier.

53. The method of claim 52, wherein the anti-HER2 monoclonal antibody is trastuzumab, pertuzumab or a biologically active variant thereof.

54. The method of claim 52, wherein the patient is a human patient.

55. The method of claim 52, wherein the cancer is a primary, secondary, refractory, or relapsing tumor.

56. The method of claim 55, wherein the primary, secondary, refractory, relapsing tumor is a sarcoma.

57. The method of claim 56, wherein the sarcoma is osteosarcoma.

58. The method of claim 55, wherein the primary, secondary, refractory, relapsing tumor is a carcinoma.

59. The method of claim 58, wherein the carcinoma is pancreatic.

60. The method of claim 59, wherein the pancreatic carcinoma is pancreatic adenocarcinoma.

61. The method of claim 52, wherein the cancer is associated with expression of HER2.

62. The method of claim 52, further comprising administering to the patient a therapeutically effective amount of radiation therapy, immunotherapy or chemotherapy or a combination thereof.

63. A method of treating a cancer, the method comprising:
(a) identifying a patient in need of treatment;
(b) administering to the patient a therapeutically effective amount of the composition of claim 50; and
(c) a pharmaceutically acceptable carrier.

64. The method of claim 63, wherein the anti-HER1 monoclonal antibody is cetuximab, panitumumab or a biologically active variant thereof.

65. The method of claim 63, wherein the patient is a human patient.

66. The method of claim 63, wherein the cancer is a primary, secondary, refractory, or relapsing tumor.

67. The method of claim 66, wherein the primary, secondary, refractory, relapsing tumor is a carcinoma.

68. The method of claim 67, wherein the carcinoma is lung cancer or colon cancer.

69. The method of claim 63, wherein the cancer is associated with expression of HER1.

70. The method of claim 63, further comprising administering to the patient a therapeutically effective amount of radiation therapy, immunotherapy or chemotherapy or a combination thereof.

71. The composition of claim 1, wherein the antibody is an anti-CD19 monoclonal antibody.

72. The composition of claim 71, wherein the anti-CD19 monoclonal antibody is blinatumomab.

73. A method of treating a cancer, the method comprising:
(a) identifying a patient in need of treatment;
(b) administering to the patient a therapeutically effective amount of the composition of claim 71; and
(c) a pharmaceutically acceptable carrier.

74. The method of claim 73, wherein the anti-CD19 monoclonal antibody is blinatumomab or a biologically active variant thereof.

75. The method of claim 73, wherein the patient is a human patient.

76. The method of claim 73, wherein the cancer is a primary, secondary, refractory, or relapsing tumor.

77. The method of claim 76, wherein the primary, secondary, refractory or relapsing tumor is a blood cell tumor.

78. The method of claim 77, wherein the blood cell tumor is lymphoma.

79. The method of claim 73, wherein the cancer is associated with expression of CD19.
